(19) United States
(12) Patent Application Publication
(10) Pub. No.: US 2020/0297209 A1
(43) Pub. Date: Sep. 24, 2020

(54) IMAGING APPARATUS AND CONTROL METHOD THEREFOR

(71) Applicant: CANON KABUSHIKI KAISHA, Tokyo (JP)

(72) Inventors: Yuji Katashiba, Kawasaki-shi (JP); Kazuhiro Matsumoto, Yokohama-shi (JP)

(21) Appl. No.: 16/899,442

(22) Filed: Jun. 11, 2020

Related U.S. Application Data
(63) Continuation of application No. PCT/JP2018/045123, filed on Dec. 7, 2018.

(30) Foreign Application Priority Data
Dec. 14, 2017 (JP) 2017-239694
Mar. 29, 2018 (JP) 2018-064217

(51) Int. Cl. A61B 3/12; A61B 3/10; A61B 3/14; A61B 3/00

(52) U.S. Cl. CPC A61B 3/1225; A61B 3/102; A61B 3/1025; A61B 3/1015; A61B 3/0025; A61B 3/14

(57) ABSTRACT

Provided is an imaging apparatus including: a first scanning unit; a second scanning unit; an optical system joining an optical path extending to the second scanning unit to an optical path extending from the second scanning unit without the second scanning unit; a common optical system illuminating the fundus via the first scanning unit and the optical system with first measuring light based on a first light source, and illuminating the fundus via the first and second scanning units with second measuring light from a second light source; a first generating unit generating a tomographic image of the fundus based on interference light of the first measuring light, and reference light based on the first light source; and a second generating unit generating a fundus image based on the second measuring light, wherein the first generating unit generates the tomographic image at a predetermined position in the fundus image.

### CROSS-REFERENCE TO RELATED APPLICATIONS

This application is a Continuation of International Patent Application No. PCT/JP2018/045123, filed Dec. 7, 2018, which claims the benefits of Japanese Patent Application No. 2017-239694, filed Dec. 14, 2017, and Japanese Patent Application No. 2018-064217, filed Mar. 29, 2018, all of which are hereby incorporated by reference herein in their entirety.

### BACKGROUND OF THE INVENTION

### Field of the Invention

The present invention relates to an imaging apparatus and a control method therefor, and more particularly, to an imaging apparatus configured to acquire a tomographic image of a retina of a fundus of an eye to be inspected (hereinafter referred to simply as “fundus”) and a control method for the imaging apparatus.

### Description of the Related Art

In particular, in recent years, such an ophthalmic imaging apparatus has been further improved in resolution in accordance with, for example, an increase in numerical aperture (NA) of an irradiation laser. However, in order to image a fundus, it is required to perform the imaging through a cornea, a crystalline lens, and other such optical tissues of the eye. Therefore, as the resolution has become higher, the aberration of the cornea and the aberration of the crystalline lens have larger influences on the quality of an acquired image.

In view of this, there is known a technology in which an adaptive optic (AO) technology is used to correct the wavefronts of measuring light and reflected light that have been disturbed by the cornea, the crystalline lens, and other such optical tissues of an eye to be inspected, to thereby acquire an image of a fine structure. In regard to OCT, there has also been developed a type of OCT that achieves a resolving power capable of resolving photoreceptor cells through use of the AO technology.

When an OCT image having a resolution high enough to allow photoreceptor cells to be resolved is acquired, the acquisition is more susceptible to a motion artifact due to the movement of the eye to be inspected than in the case of an OCT image having a normal resolution. In addition, in order to generate a tomographic image in which a plurality of B-scan images are superimposed on each other to improve image quality, it is required to acquire a plurality of B-scan images by accurately scanning the measuring light on the same line, and hence highly accurate tracking is required.

In this case, a resolving power equivalent to that of an AO-OCT image is also required for an image for movement detection, which is acquired for tracking when an AO-OCT image is acquired. Therefore, it is required to use an AO-SLO image acquired by scanning laser ophthalmoscope (AO-SLO). However, when AO-SLO and AO-OCT are implemented in a single apparatus, the entire apparatus becomes larger in size and more complicated, and also requires a large number of expensive devices, thereby leading to an increase in price. In each of Japanese Patent Application Laid-Open No. 2015-221091 and Japanese Patent No. 5641744, there is disclosed an apparatus in which AO-SLO and AO-OCT are implemented. However, in the technology disclosed in Japanese Patent Application Laid-Open No. 2015-221091, an AO-SLO image and an AO-OCT image have different imaging ranges, and there are few optical systems that can be shared, and hence the above-mentioned problem has not been solved. In the apparatus disclosed in Japanese Patent No. 5641744, imaging is not simultaneously performed by AO-SLO and AO-OCT, and it is also impossible to solve the above-mentioned problem.

### SUMMARY OF THE INVENTION

An object of the present invention is to provide an imaging apparatus capable of simultaneously acquiring an AO-OCT image and an AO-SLO image without complicating an apparatus configuration.

Another object of the present invention is to provide an imaging apparatus capable of performing tracking using an AO-SLO image when acquiring an AO-OCT image.

In order to solve the above-mentioned problem, an imaging apparatus according to at least one aspect of the present invention includes: a first scanning unit arranged to scan light on a fundus in a first direction; a second scanning unit arranged to scan the light on the fundus in a second direction being a direction different from the first direction; an optical system arranged to join an optical path extending to the second scanning unit to an optical path extending from the second scanning unit without intermediation of the second scanning unit; a common optical system arranged to: illuminate the fundus via the first scanning unit and the optical system with first measuring light obtained by branching light emitted from a first light source; and illuminate the fundus via the first scanning unit and the second scanning unit with second measuring light emitted from a second light source; a first generating unit configured to generate a tomographic image of the fundus based on interference light obtained by causing return light from the fundus of the first measuring light illuminated by the common optical system via the first scanning unit and the optical system, and reference light obtained by branching the light emitted from the first light source, to interfere with each other; and a second generating unit configured to generate a fundus image of the fundus based on return light from the fundus of the second measuring light illuminated by the common optical system via the first scanning unit and the second scanning unit, wherein the first generating unit is configured to generate the tomographic image at a predetermined position in the fundus image generated by the second generating unit.

Further, an imaging apparatus according to at least another aspect of the present invention is an image apparatus configured to acquire an image of an imaging range of a fundus, the imaging apparatus including: a first generating unit configured to generate a tomographic image of the fundus based on interference light obtained by causing return light from the fundus of first measuring light obtained by branching light emitted from a first light source, the return light being obtained when the first measuring light is scanned at a predetermined position in the imaging range, and reference light obtained by branching the light emitted from the first light source, to interfere with each other; a second generating unit configured to generate a fundus image of the fundus based on return light from the fundus of second measuring light, the return light being obtained when the second measuring light is scanned over the imaging range; and a correcting unit configured to correct irradiation positions of the first measuring light and the second measuring light on the fundus based on the fundus image generated by the second generating unit.

Further features of the present invention will become apparent from the following description of exemplary embodiments with reference to the attached drawings.

### BRIEF DESCRIPTION OF THE DRAWINGS

FIG. 1 is a view for illustrating a configuration of an imaging apparatus according to a first embodiment of the present invention.

FIG. 2 is a diagram for illustrating a display screen to be displayed on a monitor at a time of photographing in the first embodiment.

FIG. 3 is an explanatory diagram for illustrating a relationship between acquisition positions of an AO-OCT image and an AO-SLO image in the first embodiment.

FIG. 4 is an explanatory diagram for illustrating fundus tracking in the first embodiment.

FIG. 5 is an explanatory diagram for illustrating an imaging range at a time of acquiring volume data in the first embodiment.

FIG. 6 is an explanatory diagram for illustrating an imaging range at the time of acquiring the volume data in the first embodiment.

FIG. 7 is an explanatory diagram for illustrating an imaging range at the time of acquiring the volume data in the first embodiment.

FIG. 8 is an explanatory diagram for illustrating an imaging range at the time of acquiring the volume data in the first embodiment.

FIG. 9 is an explanatory diagram for illustrating an imaging range at the time of acquiring the volume data in the first embodiment.

FIG. 10 an explanatory diagram for illustrating a driving waveform of a scanner at the time of imaging in the first embodiment.

FIG. 11 an explanatory diagram for illustrating a driving waveform of a Y scanner at a time of tracking in the first embodiment.

FIG. 12 is an explanatory diagram for illustrating a driving waveform of an X scanner at the time of tracking in the first embodiment.

FIG. 13 is an explanatory view for illustrating a configuration of an X-direction scanning unit in the first embodiment.

FIG. 14 is a flow chart at the time of imaging in the first embodiment.

FIG. 15 is a flow chart at the time of tracking in the first embodiment.

FIG. 16 is a view for illustrating a schematic configuration of a fundus imaging apparatus according to a third embodiment of the present invention.

FIG. 17 is a diagram for schematically illustrating photographing ranges of an OCT optical system and an SLO optical system of the fundus imaging apparatus according to the third embodiment.

FIG. 18 is a flow chart for illustrating a fundus photographing procedure in the third embodiment.

FIG. 19 is a view for illustrating a schematic configuration of a fundus imaging apparatus according to a fourth embodiment of the present invention.

FIG. 20 is a flow chart for illustrating a fundus photographing procedure in the fourth embodiment.

### DESCRIPTION OF THE EMBODIMENTS

At least one embodiment of the present invention is described below in detail with reference to the accompanying drawings. The following description is merely illustrative and exemplary in nature, and is in no way intended to limit the present disclosure and its applications or uses. The relative arrangement of components, the steps, the numerical expressions, and the numerical values that are set forth in the at least one embodiment do not limit the scope of the present disclosure unless otherwise specifically indicated. In addition, techniques, methods, and devices that are well known by those skilled in the art are sometimes not discussed in detail because those skilled in the art are not required to know those details to achieve at least one embodiment discussed below.

### First Embodiment

Examples that can be imaged by an apparatus in a first embodiment of the present invention include a tomographic image of a retina of a fundus of an eye to be inspected.

(Apparatus Configuration)

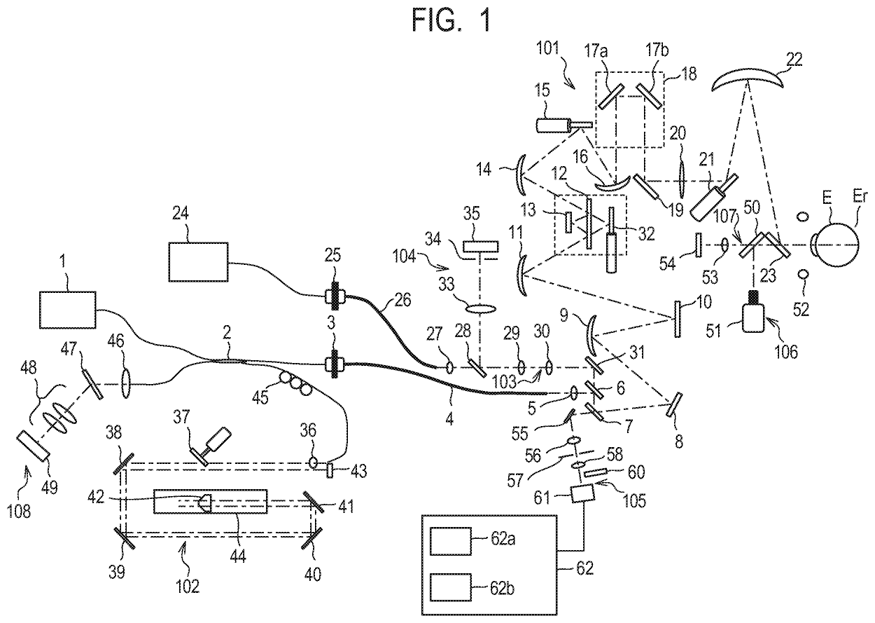

An example in which Fourier domain optical coherence tomography in the first embodiment is applied to an imaging apparatus for a fundus tomographic image is described with reference to an imaging apparatus illustrated in FIG. 1.

FIG. 1 is a view for illustrating an imaging apparatus according to the first embodiment, which includes an AO-OCT unit, an AO-SLO unit, an anterior ocular segment observation unit, and a fixation lamp unit. The imaging apparatus also includes a sample optical system being a common optical system for the AO-OCT unit and the AO-SLO unit.

A light source 1 for AO-OCT is a light source configured to generate low-coherence light as AO-OCT measuring light (first measuring light). The light source 1 functions as an example of a first light source. In the first embodiment, a super luminescent diode (SLD) light source having a center wavelength of 855 nm and a wavelength width of 100 nm is used as the light source 1.

The light emitted from the light source 1 is branched off to a sample optical system 101 and a reference optical system 102 by a fiber coupler 2. The light branched off to the sample optical system 101 side is guided to the sample optical system 101 via an adapter 3 and a fiber 4. The sample optical system 101 includes a collimator lens 5, a beam splitter (BS) 6 being a wavelength branching mirror for branching off to an AO-SLO optical system, a beam splitter (BS) 7 being a half mirror configured to branch an optical path to a wavefront detection optical system 105, a mirror 8, a concave mirror 9, a deformable mirror (DM) 10 being a shape variable mirror serving as a wavefront correction apparatus to be used for wavefront correction, a concave mirror 11, a beam splitter (BS) 12 being a wavelength branching mirror configured to branch and join OCT measuring light and SLO measuring light, a fixed mirror 13, a concave mirror 14, an X scanner 15 configured to perform scanning in the X direction, examples of which include a galvanometer mirror and a MEMS mirror, a concave mirror 16, mirrors 17a and 17b, a mirror 19, a lens 20, a Y scanner 21 configured to perform scanning in the Y direction, a concave mirror 22, and a beam splitter (BS) 23 being a wavelength selection mirror for branching off to an anterior ocular segment observation optical system 106 and a fixation lamp projection optical system 107. The mirrors 17a and 17b are integrally formed so as to be free to move in an optical axis direction by a stage 18, and perform focus adjustment on the sample optical system.

A light source 24 is a light source configured to emit AO-SLO measuring light (second measuring light). Examples of the light source 24 include a laser diode (LD) light source, an SLD light source, and a light emitting diode (LED) light source. The AO-SLO measuring light has a wavelength different from the AO-OCT measuring light. The light source 24 functions as an example of a second light source. In the first embodiment, an SLD light source having a center wavelength of 760 nm and a wavelength width of 10 nm is used. The light emitted from the light source 24 is guided to a fiber 26 via a fiber adapter 25 and to an AO-SLO projection optical system 103. In the AO-SLO projection optical system 103, the light passes through a collimator lens 27, a BS 28, lenses 29 and 30 for focus adjustment, a mirror 31, and the BS 6 to join the AO-OCT optical path of the sample optical system 101, and follows the above-mentioned path to reach the BS 12.

The BS 28 is a half mirror configured to transmit 10% of light and reflect 90% of the light in order to branch an optical path between an AO-SLO projection optical system and an AO-SLO light receiving optical system. Meanwhile, the BS 6 is a wavelength branching mirror configured to transmit the AO-SLO measuring light and reflect the AO-OCT measuring light.

As described later, the BS 12 is a wavelength branching mirror configured to reflect the AO-OCT measuring light and transmit the AO-SLO measuring light, and functions as an example of a separation unit and a joining unit. The AO-SLO measuring light is transmitted through the BS 12 to be reflected by a high-speed scanner 32 being a scanner that operates at high speed, and joins the AO-OCT measuring light by the BS 12 again. The subsequent path is shared with the AO-OCT sample optical system, and a path up to the beam splitter (BS) 23 forms the AO-SLO projection optical system. An AO-SLO light receiving optical system 104 is arranged in the reflection direction of the BS 28, and includes a lens 33, a confocal diaphragm 34, and a light-receiving element 35 formed of an avalanche photo diode (APD). The light-receiving element 35 receives return light from the fundus through the confocal diaphragm 34.

Scanners that can be used as the high-speed scanner 32 include a resonance scanner and a micro electro mechanical system (MEMS) scanner, which are capable of performing reciprocal scanning at about 8 kHz to about 16 kHz. Those high-speed scanners can adjust a swing angle, but cannot change the center angle of scanning. Therefore, it is required to prepare a separate scanner in order to perform tracking or to move an imaging range.

In addition, an irradiation position at which the AO-OCT measuring light forms an image on the fundus is adjusted so as to become substantially the same as an irradiation position at which the AO-SLO measuring light forms an image on the fundus with the high-speed scanner at a swing angle of 0.

The reference optical system 102 includes a collimator lens 36, a neutral density (ND) filter 37, mirrors 38, 39, 40, and 41, a retroreflector 42 being a corner cube mirror, and a mirror 43. The retroreflector 42 has three reflecting surfaces orthogonal to one another, and is arranged on a stage 44 so as to be movable by about ±100 mm. This allows the reference optical system 102 to handle a difference in ocular axis length of the eye to be inspected and a change in optical path length caused by moving the stage 18 of the sample optical system for the focus adjustment of the AO-OCT measuring light.

In an optical system of a spectroscope 108, interference light is caused to form an image on a line sensor 49 by a collimator lens 46, a spectral member 47 such as a grating, and an imaging lens 48.

The wavefront detection optical system 105 includes a mirror 55, a lens 56, a fundus conjugate diaphragm 57, a lens 58, a filter 60 arranged so as to be able to be inserted and retracted, and a wavefront sensor 61 serving as a wavefront detection unit, for example, a Hartmann-Shack sensor. The fundus conjugate diaphragm 57 prevents undesired light other than the return light from the fundus from entering the wavefront sensor 61.

The anterior ocular segment observation optical system 106 includes an anterior ocular segment observation camera 51, and acquires an image of an anterior ocular segment illuminated by an anterior ocular segment illumination light source 52.

The fixation lamp projection optical system 107 includes a fixation lamp presentation unit 54 such as an organic EL display device or a liquid crystal display device, and displays an indicator on the fixation lamp presentation unit 54, to thereby present a fixation target to the eye to be inspected via the lens 53 and via a BS 50 and the BS 23, each of which is a wavelength branching mirror configured to transmit visible light.

A PC 62 controls the above-mentioned units as described later.

(X-direction Scanning Unit)

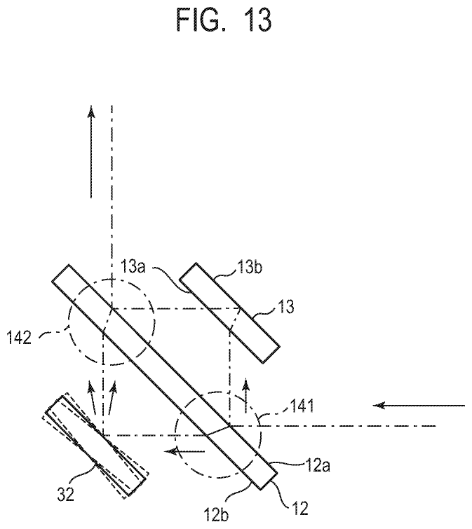

FIG. 13 is a view for illustrating a configuration of the X-direction scanning unit. The same components as those illustrated in FIG. 1 are denoted by the same reference symbols. The BS 12, which is a wavelength branching mirror, reflects AO-OCT measuring light having a wavelength of from 805 nm to 905 nm, and transmits AO-SLO measuring light having a wavelength of from 750 nm to 770 nm. This transmission characteristic is achieved by a multilayer film obtained by vacuum vapor-depositing about 30 to 70 layers of a plurality of types of dielectrics having different refractive indexes by a known method. This multilayer film is provided on a front surface on an incoming side.

The AO-OCT measuring light that has reached the multilayer film in a region 141 on the front surface 12a of the BS 12 is reflected upward in FIG. 13 to reach the fixed mirror 13. A known antireflection film is vapor-deposited on a front surface 13a of the fixed mirror 13, and its reflectance is 0.3% or smaller. Therefore, the AO-OCT measuring light is refracted by the front surface 13a, and enters a substrate to reach a back surface 13b. The substrate of the fixed mirror 13 is made of quartz glass or optical glass such as BK7, and has a refractive index of about 1.5. A metal film of, for example, silver, gold, or aluminum is vapor-deposited on the back surface 13b. The fixed mirror 13 functions as an example of a reflection unit configured to reflect the AO-OCT measuring light.

The AO-OCT measuring light is reflected by this film, is transmitted through the substrate again, exits into the air from the front surface 13a of the fixed mirror 13, reaches the multilayer film in a region 142 on the front surface 12a of the BS 12, and is reflected to join the AO-SLO measuring light. The AO-OCT measuring light is affected by astigmatism by being refracted by the front surface 13a of the fixed mirror 13 and transmitted through the substrate.

The AO-SLO measuring light is transmitted through the multilayer film in the region 141 on the front surface 12a, and is refracted to enter the substrate of the BS 12. This substrate is also made of, for example, quartz glass or optical glass such as BK7 in the same manner as the fixed mirror 13. Then, the AO-SLO measuring light is refracted again by the back surface 12b to exit into the air. The back surface 12b is provided with a known antireflection film. This antireflection film has a reflectance of 0.3% or smaller for the AO-SLO measuring light and the AO-OCT measuring light. This can reduce an occurrence of ghost and flare.

The transmitted light is affected by astigmatism by being transmitted obliquely through the BS 12 that is also a parallel plate. The AO-SLO measuring light that has exited into the air is reflected by a mirror in a movable portion of the high-speed scanner 32, and is scanned in different directions depending on the angle of the mirror in the movable portion. The mirror in the movable portion of the high-speed scanner 32 is a plane mirror, and has a surface provided with a metal film of, for example, silver, copper, or aluminum, on which a protective film being a dielectric multilayer film is provided. The AO-SLO measuring light is reflected by the surface provided with the metal film. Then, the AO-SLO measuring light is refracted by the back surface 12b (reflection blocking surface) in the region 142 of the BS 12 again, passes through the inside of the substrate, and exits into the air from the multilayer film on the front surface 12a to travel further. In this manner, the AO-SLO measuring light is transmitted through the BS 12 two times, and is therefore affected by astigmatism two times. The return light from the fundus is also affected by the same aberration in spite of traveling in the reverse direction.

At this time, the refractive indexes of the substrates of the BS 12 and the fixed mirror 13 are equalized by setting the substrates to have the same thickness and using the same glass material for the substrates. With such a configuration, the astigmatism of the AO-OCT measuring light and the astigmatism of the AO-SLO measuring light become substantially equal to each other, and highly accurate aberration correction can be performed under the same wavefront correction conditions through use of a common DM.

The incoming optical path for the AO-OCT measuring light and the AO-SLO measuring light to the X-direction scanning unit serves as the outgoing optical path for the return light of the AO-OCT measuring light and the return light of the AO-SLO measuring light from the X-direction scanning unit. In the same manner, the outgoing optical path for the AO-OCT measuring light and the AO-SLO measuring light from the X-direction scanning unit serves as the incoming optical path for the return light of the AO-OCT measuring light and the return light of the AO-SLO measuring light to the X-direction scanning unit.

In addition, intervals and angles among the high-speed scanner 32, the BS 12, and the fixed mirror 13 are adjusted so that the DM 10, the wavefront sensor 61, the X scanner 15, the Y scanner 21, and the pupil of the eye to be inspected become conjugate with the AO-OCT measuring light and the AO-SLO measuring light with the projection positions of the fundus matching each other. This allows the aberration of the AO-OCT measuring light and the aberration of the AO-SLO measuring light to be satisfactorily corrected by one DM 10.

With the above-mentioned configuration, it is possible to obtain a front image of the fundus centering on the imaging range of the AO-OCT image without affecting the light flux of the AO-OCT measuring light. Therefore, it is possible to perform highly accurate tracking through use of the front image.

(Imaging Method)

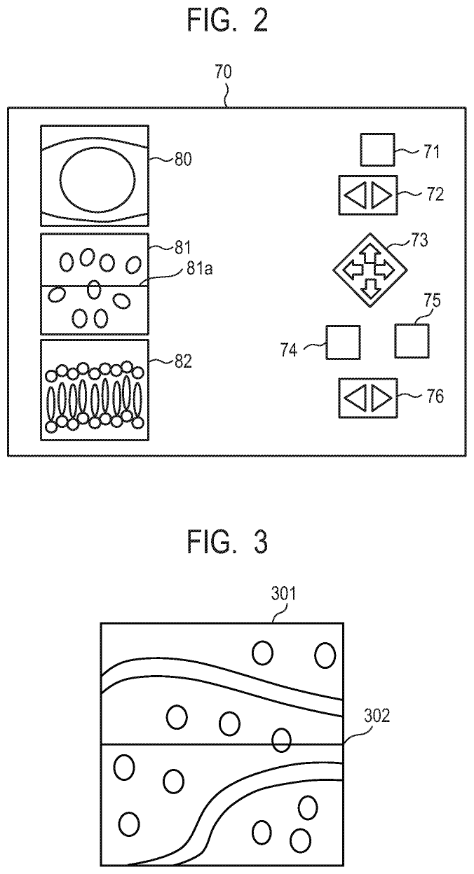

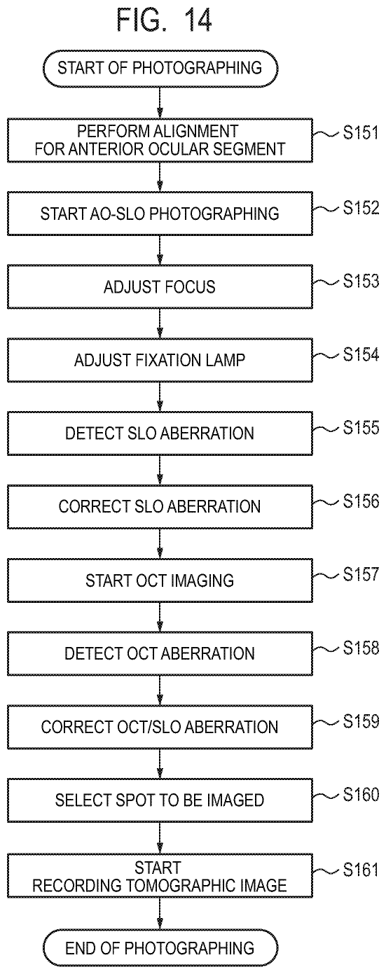

Next, a method of acquiring an AO-OCT image for creating a superimposed image by scanning the same region of a fundus Er of an eye E to be inspected a plurality of times through use of the apparatus having the above-mentioned configuration is described with reference to FIG. 2 and FIG. 14. FIG. 2 is a diagram for illustrating a display screen to be displayed on a monitor of the PC 62 at a time of imaging, and FIG. 14 is a flow chart at the time of imaging.

The eye E to be inspected is placed in front of this apparatus. The reflected light from an anterior ocular segment of the eye to be inspected, which is illuminated by the anterior ocular segment illumination light source 52, is reflected by the BS 50, and imaged by the anterior ocular segment observation camera 51 to be displayed in a display area 80 for an anterior ocular segment image on a monitor 70 of the PC 62. While viewing this anterior ocular segment image, a person who performs imaging (hereinafter referred to as “imaging person”) uses an alignment mechanism (not shown) to align the position of the optical system with the position of the pupil of the eye to be inspected so that the center of a pupil image of the eye to be inspected is located at the center of the display area 80 and an iris pattern can be clearly viewed (Step S151).

When the alignment is finished, the imaging person operates a start switch 71 for starting to acquire an AO-SLO image on the monitor. The PC 62, which has detected the input to the start switch 71, turns on the light source 24 for AO-SLO, and drives the high-speed scanner 32 and the Y scanner 21 to start raster scanning of the AO-SLO measuring light on the fundus (Step S152).

The light reflected and scattered by the fundus Er illuminated in the above-mentioned manner returns through the AO-SLO sample optical system, and 10% of the light is transmitted through the BS 7, and passes through the fundus conjugate diaphragm 57 to reach the wavefront sensor 61. 90% of the light reflected by the BS 7 is transmitted through the BS 6, and 80% of the light is reflected by the BS 28, and condensed on the confocal diaphragm 34 by the lens 33. The light passing through the confocal diaphragm 34 reaches the light-receiving element 35 formed of, for example, an APD or a PMT. The PC 62 generates image data based on a voltage signal corresponding to the amount of light received by the light-receiving element 35, and the image data is displayed in a display area 81 for an AO-SLO image on the monitor 70 as a front image of the fundus. Therefore, the PC 62 functions as an example of a second generating unit configured to generate a front image of the fundus.

While viewing this image, the imaging person operates a focus switch 72 to perform focus adjustment. The PC 62, which has detected the input to the focus switch 72, controls a drive unit of the stage 18 to move the mirrors 17a and 17b integrally in the optical axis direction. With this control, a fundus-conjugate position in the optical system changes, to thereby be able to adjust the focus at a desired depth position (Step S153). This is called “Badal optical system”, in which an image formation relationship for the pupil is maintained. In this manner, an AO-SLO image at a desired depth is displayed in the display area 81.

In addition, the imaging person operates the fixation lamp operation switch 73 to guide the fixation of the eye to be inspected so that a fundus image of a desired region is imaged (Step S154).

(Aberration Correction)

When detecting that an AO-SLO image having a signal intensity equal to or higher than a certain level is stably obtained, the PC 62 starts an aberration correction (AO) operation. A part of the reflected light from the fundus is transmitted through the BS 7 to reach the wavefront sensor 61, and output data from the wavefront sensor 61 is sent to the PC 62. The wavefront sensor 61 has a lens array arranged in front of a light-receiving element, and hence an output image from the wavefront sensor 61 is an image in which spots are arranged in a grid pattern (lattice pattern). When the eye to be inspected exhibits aberration, those spots change in position. The direction and amount of deviation of each spot are analyzed to obtain wavefront aberration (Step S155).

In order to correct this wavefront aberration, the PC 62 obtains a pattern for displacing each of micromirrors forming the DM 10 serving as an aberration correction device. The PC 62 controls the driving of the micromirrors of the DM 10 based on this pattern to correct the aberration (Step S156).

Due to the aberration correction of the AO-SLO measuring light, the diameter of a spot projected on the fundus becomes smaller, and at the same time, the diameter of a spot of which an image is formed on a confocal diaphragm also becomes smaller, to thereby increase the amount of light received by the light-receiving element 35. Therefore, a high-resolution image of photoreceptor cells is clearly displayed in the display area 81 of the AO-SLO image on the monitor 70 of the PC 62. A line 81a indicates an imaging position of an AO-OCT image.

(AO-OCT Imaging)

Next, the imaging person operates a switch 74 to give an instruction to start imaging for acquiring an AO-OCT image (Step S157). The PC 62, which has detected the input to the switch 74, turns on the light source 1 for AO-OCT. Thus, the AO-OCT measuring light branched off to the sample optical system side by the fiber coupler 2 enters the sample optical system from the fiber 4. The AO-OCT measuring light that has entered the sample optical system is collimated by the collimator lens 5 to reach the BS 12. As described above, the AO-OCT measuring light is reflected by the BS 12, further reflected by the fixed mirror 13, and again reflected by the BS 12, to thereby join the optical path for the AO-SLO measuring light. Thus, the AO-OCT measuring light reaches the eye to be inspected without being affected by the high-speed scanner 32. The reflected light of the AO-OCT measuring light by the fundus follows back the sample optical system, and is reflected by the BS 12, reflected by the fixed mirror 13, and again reflected by the BS 12 to reach the BS 7 being a half mirror. The BS 7 has transmission characteristics of reflecting 90% of light and transmitting 10% of light. 10% of the light reflected by the BS 7 is transmitted to travel to the wavefront sensor 61. The remaining 90% of the reflected light is reflected by the BS 7 and the BS 6, and an image of the reflected light is formed on an end face of the fiber 4 by the collimator lens 5. Then, the reflected light is transmitted through the fiber 4 to reach the fiber coupler 2.

(AO-OCT Wavefront Detection)

When the imaging for acquiring an AO-OCT image is started, the filter 60 for cutting off the AO-SLO measuring light is inserted in front of the wavefront sensor 61. Therefore, the AO-OCT measuring light is the only light that reaches the wavefront sensor 61, and the light of which the wavefront is detected is switched from the AO-SLO measuring light to the AO-OCT measuring light, to thereby detect the wavefront aberration of the AO-OCT measuring light (Step S158). However, the AO-SLO projection optical system includes the lenses 29 and 30 for focus adjustment, and a difference in focus position due to a difference in wavelength between the AO-SLO measuring light and the AO-OCT measuring light is automatically corrected by the lenses 29 and 30 for focus adjustment. In the same manner as described above, the driving amount of each micromirror of the DM 10 is calculated based on the detected wavefront aberration to drive the DM 10, to thereby correct the wavefront aberration (Step S159). Through this correction, the AO-OCT image displayed in a display area 82 becomes brighter with an improved contrast.

(Reference Optical System)

Reference light branched off to the reference optical system side by the fiber coupler 2 has its polarization adjusted by a polarization adjuster 45 so as to have its polarization state matching the return light from the sample optical system, and enters the reference optical system 102 to be adjusted to have an appropriate light amount by the neutral density (ND) filter 37 for adjustment of a reference light amount. Then, the reference light reflected by the mirrors 38, 39, 40, and 41 to be reflected by the retroreflector 42 is reflected by the mirrors 41, 40, 39, and 38 to reach the mirror 43. The light vertically reflected by the mirror 43 again follows back the optical path to return to the fiber coupler 2.

This reference light from the reference optical system 102 and the return light from the fundus of the eye to be inspected in the sample optical system are joined (combined) by the fiber coupler 2, and guided to the spectroscope 108 as interference light. The interference light guided to the spectroscope 108 is collimated by the collimator lens 46, and spectrally dispersed by the spectral member 47 such as a diffraction grating, and an image of an interference wave is formed on the line sensor 49 by the lens 48. Output from the line sensor 49 is sent to the PC 62, and converted into digital data by an A/D converter 62a to be stored in a memory 62b. This data is subjected to removal of fixed pattern noise and wave number conversion, and then calculated by a known method such as frequency analysis, to thereby generate tomographic image data. Therefore, the PC 62 functions as an example of a first generating unit configured to generate a tomographic image of the fundus.

(Gate Adjustment)

The imaging person uses an optical path length adjustment switch 76 illustrated in FIG. 2 to adjust the optical path length of the reference optical system 102 so that a desired tomographic image is displayed in a display area. The PC 62, which has detected the input to the optical path length adjustment switch 76, drives the stage 44 in a direction corresponding to the input. Thus, the retroreflector 42 moves in the optical axis direction, to thereby change the optical path length. When the optical path length of the sample optical system and the optical path length of the reference optical system substantially match each other, a tomographic image of photoreceptor cells is displayed in the display area 82 (Step S160).

(Scanning)

At this time, the AO-SLO image acquired simultaneously is displayed in the display area 81. The high-speed scanner 32 is arranged so as to bypass an OCT optical system, and hence the AO-OCT measuring light is only line-scanned in the Y direction (in FIG. 3, the horizontal direction or a first direction) by the Y scanner 21. The AO-SLO measuring light is scanned in the Y direction by the Y scanner 21, and scanned in the X direction (in FIG. 3, the vertical direction or a second direction) by the high-speed scanner 32. Therefore, as illustrated in FIG. 3, an AO-SLO image of an imaging range 301 centered on a scanning line 302, which is the acquisition position of the AO-OCT image, in the up-down direction is acquired (Step S161). The first direction may be the X direction, and the second direction may be the Y direction.

In FIG. 10, a waveform 111 indicates the scanning angle of the mirror of the high-speed scanner 32, the horizontal axis represents a time, and the vertical axis represents an angle. That is, the high-speed scanner 32 reciprocates at high speed in the X direction. At the same time, as indicated by a waveform 112, the Y scanner 21 is moved for scanning at a cycle period slower than that indicated by the waveform 111. The image acquisition is performed on both the outward path and the return path of the high-speed scanner.

In FIG. 10, a period P1 indicates a period for acquiring one frame of an AO-OCT image and one frame of an AO-SLO image, and a period P2 indicates a retrace period of the Y scanner 21, in which the image acquisition is not performed. That is, the Y scanner 21 continuously changes in angle from θ2 to −θ2 in the period P1, and is moved for scanning from −θ2 to +θ2 in the period P2 at an angular velocity higher than the angular velocity exhibited in the period P1. Through the repetition of such scanning, the imaging of a two-dimensional region is performed by the AO-SLO unit, and the imaging of a line is performed by the AO-OCT unit.

(Tracking)

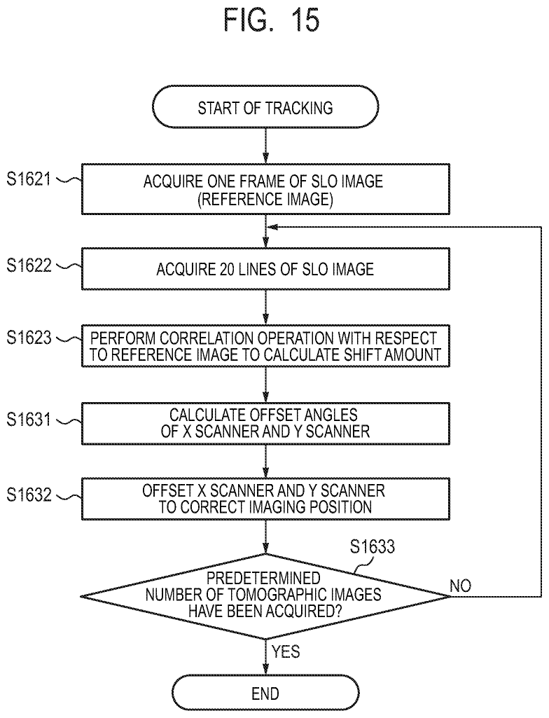

Tracking involving the movement detection of the eye to be inspected and the correction of the imaging range is described with reference to a flow chart illustrated in FIG. 15.

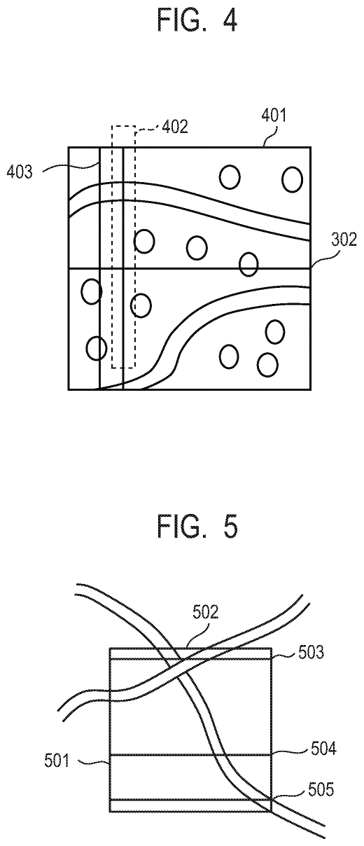

When the imaging for acquiring an AO-OCT image is started, one frame of an AO-SLO image for the imaging range 301 is recorded as a tracking reference image as illustrated in FIG. 3 (Step S1621). At this time, the tracking reference image has an image size of 400×400 pixels, and is formed of 400 vertical lines. Assuming that the high-speed scanner 32 is set to have a scan frequency of 16 kHz, it takes 12.5 msec to acquire an image formed of 400 lines due to the reciprocating manner of image acquisition, and images of about 70 frames are obtained per second. As illustrated in FIG. 4, during the imaging of the next frame, every time an image at a predetermined line is acquired, a correlation operation is performed with respect to a reference image 401 to detect the movement of the eye to be inspected. For example, in the first embodiment, a tracking image 402 is created from signals corresponding to 20 lines (Step S1622). This enables position detection to be performed 20 times (=400/20) during the scanning along one line of an AO-OCT image. That is, the movement of the eye to be inspected can be detected and corrected in a time period of 1 msec or less. Therefore, even an involuntary eye movement during fixation at a speed of about 1 mm per second can be tracked with an accuracy of 1 μm or less, and it is possible to obtain sufficient accuracy for OCT line scanning with a line width of 3 μm. As soon as each tracking image is acquired, a correlation operation is performed with respect to the reference image 401 to obtain a shift amount (sf302_X, SF302_Y) (Step S1623). In this case, FIG. 4 is an illustration of an image 403 corresponding to the tracking image 402 in the reference image 401. When there is no movement of the eye to be inspected, the shift amount of the N-th tracking image is (x, y)=(20×(n−1), 0), and hence a movement amount of the eye to be inspected, which is obtained from the N-th tracking image, is (sf302_x-20N, SF302_y) (S1623).

The mirror rotation angles of the X scanner 15 and the Y scanner 21 are calculated from this movement amount (Step S1631), and the X scanner 15 and the Y scanner 21 are driven so as to follow the movement of the eye to be inspected, which has been obtained as described above. The Y scanner 21 is being driven with a predetermined waveform, and hence the drive center is offset. In addition, the X scanner 15 is only offset by the obtained amount (Step S1632).

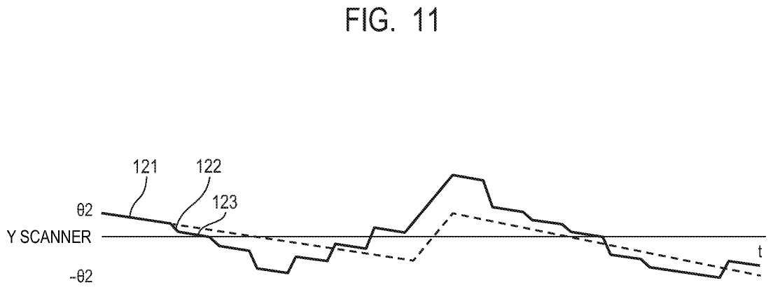

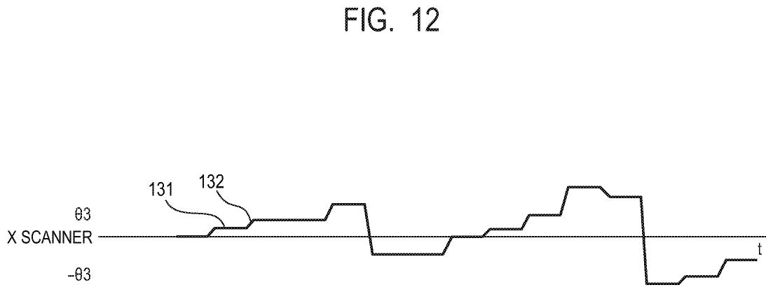

In FIG. 11, a waveform 121 indicates a change in angle of the Y scanner 21. In FIG. 12, a waveform 131 indicates a change in angle of the X scanner 15. In FIG. 11 and FIG. 12, the horizontal axis represents a time, and the vertical axis represents a scanning angle. As described above, the shift amount of the angle for correcting the movement amount of the eye to be inspected is calculated at regular intervals, and hence the scanning angle is shifted as indicated by a waveform 122. After that, scanning is performed at a normal angular velocity as indicated by a waveform 123. The solid line indicates a mirror angle subjected to an angle shift, and the broken line indicates a mirror angle subjected to no angle shift. The waveform 131 of FIG. 12 indicates a change in angle of the X scanner 15, and the horizontal axis represents a time. The X scanner 15 is used only for tracking to correct the movement of the eye to be inspected, and hence the angle changes only by the calculated offset amount.

The AO-SLO image is acquired even during the driving of the X scanner 15 as indicated by a waveform 132, but the correlation operation is performed on an image excluding the relevant portion. Through the continuation of such tracking control, the AO-OCT unit can obtain a plurality of, namely, a predetermined number of, for example, about 100 tomographic images on the same line (Step S1633). Those tomographic images are acquired for exactly the same region, and it is possible to create an AO-OCT image having a high contrast by superimposing those tomographic images.

(OCT Volume Scan)

When an AO-OCT volume scan switch 75 is operated, scanning in a volume scan mode is started.

When the imaging person operates the AO-OCT volume scan switch 75 to set an imaging range through use of a cursor (not shown), the PC 62 also drives the Y scanner 21 to shift an OCT line scanning line in the Y direction in synchronization with the X scanner 15.

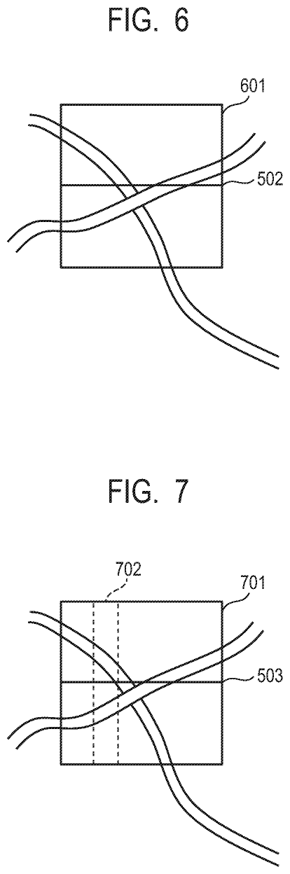

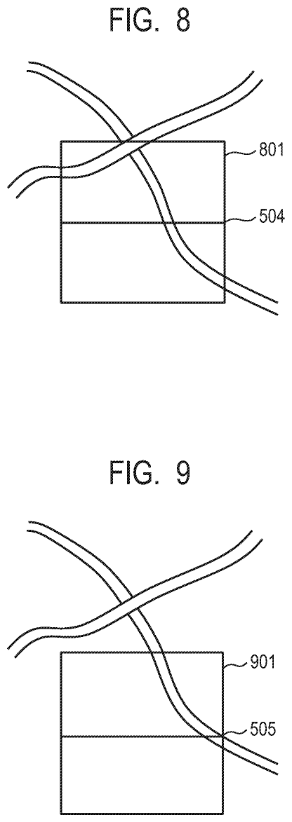

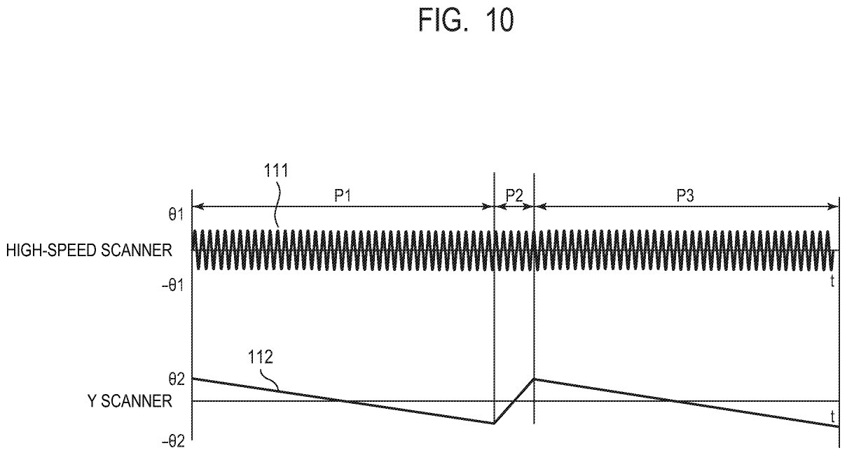

In FIG. 5, a frame 501 on the fundus image indicates an imaging range of an AO-OCT volume scan. The AO-OCT measuring light is scanned over a region corresponding to the frame 501 on the fundus image to acquire AO-OCT volume data. In FIG. 6, a frame 601 (hereinafter referred to as “imaging range 601”) indicates an imaging range of an AO-SLO image used when the AO-OCT measuring light is scanned along an imaging line 502, which is the uppermost imaging line in the imaging range of FIG. 5. As illustrated in FIG. 6, the imaging range of the AO-SLO image is a rectangular region centered on the imaging line 502 of the AO-OCT image. The AO-SLO image obtained for the imaging range is stored as a tracking reference image. Subsequently, the X scanner 15 is driven to start to scan the AO-OCT measuring light along the next imaging line indicated by a line 503 of FIG. 5. An imaging range of the AO-SLO image at that time is indicated by a frame 701 (hereinafter referred to as “imaging range 701”) in FIG. 7. The imaging range 701 of the AO-SLO image has moved downward from the imaging range 601 by one line of the AO-OCT image. That is, when an AO-OCT image is being acquired along a line 504 of FIG. 5, the AO-SLO image is acquired for an imaging range indicated by a frame 801 in FIG. 8, and a frame 901 of FIG. 9 is an imaging range of the AO-SLO image when the imaging is being performed along a line 505, which is the lowermost line. In this manner, the imaging range of the AO-SLO image moves in accordance with the movement of the scan line of the AO-OCT measuring light. Therefore, image information on a neighboring region around the imaging range of the AO-OCT image is obtained at all times.

(Tracking)

As described above, the AO-SLO image is used to perform tracking as well at the time of the AO-OCT volume scan. During the scanning along the line 503 of FIG. 7, an image corresponding to a range of 20 lines indicated by the broken line is extracted while an image for the imaging range 701 of the AO-SLO image is being acquired, and is subjected to the correlation operation with respect to the image for the imaging range 601 of the AO-SLO image acquired last time, to thereby obtain a shift amount. In the same manner as described above, the shift amounts of the X scanner 15 and the Y scanner 21 are calculated from the shift amount obtained above, and the X scanner 15 and the Y scanner 21 are immediately driven to perform fundus tracking for correcting the irradiation positions of the AO-OCT measuring light and the AO-SLO measuring light on the fundus.

Through the repetition of the line scanning while performing tracking in such a manner, AO-OCT images (volume data) are acquired for the imaging range 501 of the AO-OCT image. Thus, stereoscopic information on the region corresponding to the imaging range 501 of FIG. 5 is obtained.

The AO-SLO images used for tracking may each be corrected by the shift amount to create one reference image, and may be used as the reference image when the line scanning for an AO-OCT image is performed along the next imaging line. This can always keep the reference image updated, to thereby be able to perform more accurate tracking.

### Second Embodiment

In the first embodiment, the configuration in which the AO-OCT image is acquired at a predetermined position (center position in the X direction) of the imaging range of the AO-SLO image has been described. In a second embodiment of the present invention, a case in which the fixed mirror 13 illustrated in FIG. 13 is replaced by a galvanometer mirror is described.

The galvanometer mirror is changed in angle by an angle corresponding to one line (in the X direction) each time one AO-SLO image (one frame) is acquired. Thus, the AO-OCT volume data for the imaging range can be acquired. The range for which the AO-SLO image is acquired does not change, and hence it is not required to take into consideration a shift amount when a frame is acquired during tracking.

### Third Embodiment

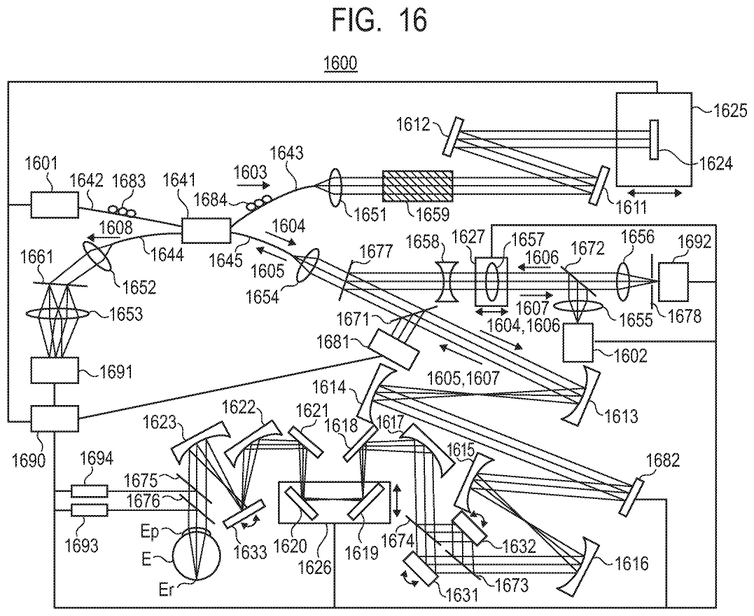

A fundus imaging apparatus according to a third embodiment of the present invention, which is used for acquiring an image of, for example, a fundus of an eye to be inspected, is described below in detail with reference to FIG. 16 to FIG. 18.

(Apparatus Configuration)

A fundus imaging apparatus 1600 serving as one mode of the fundus imaging apparatus according to the third embodiment is described with reference to FIG. 16. The fundus imaging apparatus 1600 according to the third embodiment includes an OCT optical system, an SLO optical system, an anterior ocular observation optical system, a fixation lamp optical system, and a control unit 1690. In the third embodiment, the entire optical system is mainly formed of a reflective optical system using mirrors.

The control unit 1690 may be configured through use of a general-purpose computer, or may be configured as a computer dedicated to the fundus imaging apparatus 1600. The control unit 1690 may be configured separately from, or may be integrally with, an imaging unit including the OCT optical system, the SLO optical system, the anterior ocular observation optical system, and the fixation lamp optical system.

First, the OCT optical system of the fundus imaging apparatus 1600 is described. A light source 1601 is a light source for generating light (low-coherence light). In the third embodiment, a super luminescent diode (SLD) having a center wavelength of 830 nm and a bandwidth of 50 nm is used as the light source 1601. The SLD is selected in the third embodiment, but any type of light source that can emit low-coherence light may be used, and it is also possible to use, for example, an amplified spontaneous emission (ASE). The light source 1601 is connected to the control unit 1690 so as to be controlled by the control unit 1690.

The wavelength of light emitted from the light source 1601 can also be set to a wavelength corresponding to near-infrared light in consideration of the measuring of an eye. In addition, the wavelength of light emitted from the light source 1601 affects a resolution power in the lateral direction of the obtained tomographic image, and can therefore be set as short as possible. In the third embodiment, the center wavelength is set to 830 nm. Another wavelength may be selected depending on a spot to be measured of an object to be observed. In addition, as the wavelength band becomes wider, the resolution power in the depth direction becomes more satisfactory. In general, when the center wavelength is 830 nm, the resolution power is 6 μm in a bandwidth of 50 nm, and the resolution power is 3 μm in a bandwidth of 100 nm. The center wavelength and the bandwidth of the light source 1601 are not limited thereto, and may be changed depending on a desired configuration.

The light emitted from the light source 1601 passes through a single mode fiber 1642 to be guided to an optical coupler 1641 serving as a light splitting unit. The light emitted from the light source 1601 is split by the optical coupler 1641 at an intensity ratio of 90:10, and becomes reference light 1603 and OCT measuring light 1604, respectively. The splitting ratio is not limited thereto, and can be appropriately selected depending on an object to be inspected.

Next, an optical path for the reference light 1603 is described. The reference light 1603 obtained through the splitting by the optical coupler 1641 passes through a single mode fiber 1643 to be guided to the lens 1651, and is emitted as collimated light. Subsequently, the reference light 1603 is transmitted through a glass 1659 for dispersion compensation to be guided by mirrors 1611 and 1612 to a mirror 1624 being a reference mirror. In the third embodiment, a plane mirror is used as a reference mirror. The fight reflected by the mirror 1624 is again reflected by the mirror 1612 and the mirror 1611 in the stated order, and transmitted through the glass 1659 for dispersion compensation to be guided to the optical coupler 1641.

The glass 1659 for dispersion compensation can compensate dispersion obtained when the OCT measuring light 1604 reciprocates between the eye E to be inspected and a lens 1654 with respect to the reference light 1603.

The mirror 1624 is mounted on an electric stage 1625, and serves as an optical path length adjusting unit. The electric stage 1625 can be moved in an optical axis direction of the reference light 1603 as indicated by the arrow in FIG. 16, and it is possible to adjust the optical path length of the reference light 1603 by moving the position of the mirror 1624. The electric stage 1625 is controlled by the control unit 1690.

Next, an optical path for the OCT measuring light 1604 is described. The OCT measuring light 1604 obtained through the splitting by the optical coupler 1641 passes through a single mode fiber 1645 to be guided to the lens 1654, and is emitted as collimated fight.

Subsequently, the OCT measuring light 1604 is transmitted through a dichroic mirror 1677 and a beam splitter 1671, and reflected by mirrors 1613 and 1614 to enter a deformable mirror 1682 being an aberration correcting unit. In this case, the deformable mirror 1682 is a mirror device configured to correct aberration between the OCT measuring light 1604 and an OCT return light 1605 by freely deforming its mirror shape based on aberration detected by a wavefront sensor 1681 being aberration measuring unit.

In the third embodiment, a deformable mirror is used as the aberration correcting unit, but the aberration correcting unit is only required to be able to correct aberration, and it is also possible to use, for example, a spatial light phase modulator using liquid crystal. In addition, in the third embodiment, a Shack-Hartmann wavefront sensor 1681 is used as the aberration measuring unit. However, the aberration measuring unit is not limited thereto, and may be configured through use of any known sensor or the like for measuring aberration. The deformable mirror 1682 and the wavefront sensor 1681 are controlled by the control unit 1690.

After being reflected by the deformable mirror 1682, the OCT measuring light 1604 is reflected by mirrors 1615 and 1616 to enter a dichroic mirror 1673. In this case, the dichroic mirror 1673 and a dichroic mirror 1674 reflect light from the light source 1601 and transmit light from a light source 1602 depending on the wavelength of the light.

The OCT measuring light 1604 reflected by the dichroic mirror 1673 enters an X scanner 1632 (second scanning unit). The center of the OCT measuring light 1604 is adjusted so as to match the rotation center of the X scanner 1632, and through the rotation of the X scanner 1632, it is possible to scan the OCT measuring light 1604 on a retina Er of the eye E to be inspected in a direction perpendicular to the optical axis. In this case, a galvanometer mirror is used as the X scanner 1632. The X scanner 1632 may be formed of any other deflecting mirror. Although not shown, the X scanner 1632 is connected to the control unit 1690 so as to be controlled by the control unit 1690.

The OCT measuring light 1604 reflected by the X scanner 1632 is reflected by the dichroic mirror 1674, and then reflected by mirrors 1617 to 1620 in the stated order.

The mirrors 1619 and 1620 are mounted on an electric stage 1626, and form a first focus unit. The electric stage 1626 can be moved in a direction toward or away from the mirror 1618 and a mirror 1621 as indicated by the arrow in FIG. 16. The electric stage 1626 is controlled by the control unit 1690. The mirrors 1619 and 1620 are arranged within a common optical path of the OCT optical system and the SLO optical system. Therefore, when the mirrors 1619 and 1620 are moved by the electric stage 1626, focus states of the OCT measuring light 1604 and SLO measuring light 1606 can be adjusted in correspondence with a diopter of the eye E to be inspected.

In the third embodiment, a moving range of the electric stage 1626 is set to 1,660 mm, and it is possible to adjust focus positions of the OCT measuring light 1604 and the SLO measuring light 1606 in correspondence with a diopter range of from −12D to +7D of the eye E to be inspected. The moving range of the electric stage 1626 may be freely set by a desired configuration.

In this case, in the third embodiment, the first focus unit arranged on the common optical path of the OCT optical system and the SLO optical system is formed of a Badal optical system including a reflective optical system of mirrors 1619 and 1620. The use of the reflective optical system can prevent undesired stray light from entering the wavefront sensor 1681, and it is possible to measure and correct aberration with accuracy.

The OCT measuring light 1604 reflected by the mirror 1620 is reflected by the mirror 1621 and a mirror 1622 to enter a Y scanner 1633 (first scanning unit). The center of the OCT measuring light 1604 is adjusted so as to match the rotation center of the Y scanner 1633, and through the rotation of the Y scanner 1633, it is possible to scan the OCT measuring light 1604 on the retina Er in a direction perpendicular to the optical axis and to the scanning direction of the X scanner 1632. In this case, a galvanometer mirror is used as the Y scanner 1633. The Y scanner 1633 may be formed of any other deflecting mirror.

Although not shown, the Y scanner 1633 is connected to the control unit 1690 so as to be controlled by the control unit 1690. The X scanner 1632 and the Y scanner 1633 form an OCT scanning unit configured to scan the OCT measuring light 1604 on the fundus of the eye E to be inspected in a two-dimensional direction.

The OCT measuring light 1604 reflected by the Y scanner 1633 is reflected by a mirror 1623, and transmitted through dichroic mirrors 1675 and 1676 to enter the eye E to be inspected. The X scanner 1632, the Y scanner 1633, and the mirrors 1617 to 1623 function as an optical system for scanning the OCT measuring light 1604 on the retina Er. With this optical system, it is possible to scan the OCT measuring light 1604 on the retina Er with a pupil Ep being used as a fulcrum.

The OCT measuring light 1604, which has entered the eye E to be inspected, is reflected or scattered by the retina Er, and returns as the OCT return light 1605 along the optical path for the OCT measuring light 1604 to be guided again to the optical coupler 1641.

The reference light 1603 and the OCT return light 1605 are combined by the optical coupler 1641 to become interference light. In this case, when the optical path length of the OCT measuring light 1604 and the OCT return light 1605 and the optical path length of the reference light 1603 are substantially equal to each other, the OCT return light 1605 and the reference light 1603 interfere with each other to become interference light. The control unit 1690 controls the electric stage 1625 to move the mirror 1624, to thereby be able to match the optical path length of the reference light 1603 with the optical path length of the OCT measuring light 1604 and the OCT return light 1605, which changes depending on a measurement target part of the eye E to be inspected. Combined light 1608 (interference light) is emitted from a single mode fiber 1644 as spatial light, and passes through a lens 1652 to be guided to a transmissive grating 1661. After that, the light 1608 is spectrally dispersed for each wavelength by the transmissive grating 1661, and condensed by a lens 1653 to enter a line camera 1691.

The light 1608 that has entered the line camera 1691 is converted into a voltage signal (interference signal) corresponding to a light intensity for each position (wavelength) on the line camera 1691. Specifically, an interference pattern in a spectral region on a wavelength axis is observed on the line camera 1691. The obtained voltage signal group is converted into a digital value. The control unit 1690 can generate a tomographic image of the eye E to be inspected by performing data processing on the interference signal converted into the digital value. The control unit 1690 also displays the generated tomographic image on a display unit (not shown). The display unit may be formed of any monitor, and may be configured separately from, or may be integrally with, the imaging unit and the control unit 1690. In addition, the data processing to be performed when a tomographic image is generated may be any known data processing for generating a tomographic image from an interference signal.

The single mode fibers 1642 and 1643 are provided with paddles 1683 and 1684 for polarization adjustment, respectively. The paddles 1683 and 1684 for polarization adjustment can adjust the polarization of light passing through the single mode fibers 1642 and 1643, respectively. Through use of the paddles 1683 and 1684 for polarization adjustment, the polarization state of the light from the light source 1601 can be adjusted, and the polarization of the reference light 1603 can be adjusted so that the polarization states of the OCT return light 1605 and the reference light 1603 match each other. The position at which a paddle for polarization adjustment is provided is not limited thereto, and the paddle for polarization adjustment may be provided on, for example, the single mode fiber 1645 or the like.

Incidentally, the OCT return light 1605 is split by the beam splitter 1671 while returning along the optical path for the OCT measuring light 1604, and a part of the OCT return light 1605 enters the wavefront sensor 1681. The wavefront sensor 1681 measures the aberration of the OCT return light 1605 that has entered the wavefront sensor 1681. In the third embodiment, the beam splitter 1671 reflects a part of the OCT return light 1605 and transmits an SLO return light 1607 described later. This allows the aberration of the OCT return light 1605 to be selectively measured. The wavefront sensor 1681 is electrically connected to the control unit 1690. The control unit 1690 substitutes output from the wavefront sensor 1681 into a Zernike polynomial, to thereby grasp the aberration of the eye E to be inspected, which has been measured by the wavefront sensor 1681.

In regard to a defocus component of the Zernike polynomial, the control unit 1690 controls the positions of the mirrors 1619 and 1620 through use of the electric stage 1626 to correct the diopter of the eye E to be inspected. The control unit 1690 also controls the surface shape of the deformable mirror 1682 to correct a component other than the defocus component. This enables the control unit 1690 to generate (acquire) a tomographic image having a high lateral resolution power.

In this case, the mirrors 1613 to 1623 are arranged so that the pupil Ep, the X scanner 1632, the Y scanner 1633, the wavefront sensor 1681, and the deformable mirror 1682 become optically conjugate. This enables the wavefront sensor 1681 to measure the aberration of the eye E to be inspected.

Next, the SLO optical system is described. The light source 1602 is a light source for generating light having a wavelength different from that of the light source 1601. In the third embodiment, an SLD having a wavelength of 780 nm is used as the light source 1602. The type of the light source 1602 for the SLO optical system is not limited thereto, and it is also possible to use, for example, a laser diode (LD) as the light source 1602. In addition, the wavelength of the light source 1602 is not limited thereto, and may be changed depending on a desired configuration. The light source 1602 is connected to the control unit 1690 so as to be controlled by the control unit 1690.

The light emitted from light source 1602 is guided to a lens 1655 to be emitted as collimated light. The light transmitted through the lens 1655 is guided to a beam splitter 1672 to be split at an intensity ratio of 90:10 between the transmitted light and the reflected light (SLO measuring light 1606). The SLO measuring light 1606 reflected by the beam splitter 1672 is transmitted through a focus lens 1657 and a lens 1658.

The focus lens 1657 is mounted on an electric stage 1627, and serves as a second focus unit. The electric stage 1627 can be moved in an optical axis direction of the SLO measuring light 1606 as indicated by the arrow in FIG. 16, and it is possible to adjust the focus state of the SLO measuring light 1606. The electric stage 1627 is controlled by the control unit 1690.

The control unit 1690 controls the electric stage 1627 to move the focus lens 1657, to thereby be able to adjust the focus position of the SLO measuring light 1606 to a position different from the focus position of the OCT measuring light 1604. In this case, a moving range of the electric stage 1627 is set to 10 mm, and corresponds to a diopter range of from −2D to +2D. The moving range of the electric stage 1627 is not limited thereto, and may be set to any moving range that is smaller than the moving range of the electric stage 1626.

In the third embodiment, the first focus unit is used to perform the focus adjustment of the OCT measuring light 1604 and the SLO measuring light 1606 and correct the diopter of the eye E to be inspected, and hence a focus adjustment range of the second focus unit can be kept narrow. It is thus possible to cause the moving range of the electric stage 1627 to become narrower than the moving range of the electric stage 1626. Therefore, the focus positions of the OCT optical system and the SLO optical system can be adjusted to mutually different positions through use of a smaller stage, to thereby be able to reduce the size of the optical systems.

In FIG. 16, the focus lens 1657 is illustrated as a convex lens, and the lens 1658 is illustrated as a concave lens, but the configurations of the focus lens 1657 and the lens 1658 are not limited thereto. The focus lens 1657 and the lens 1658 may be configured as a concave lens and a convex lens, respectively, or may be both configured as convex lenses so as to form an intermediate image therebetween.

The light transmitted through the focus lens 1657 and the lens 1658 travels to the dichroic mirror 1677. The dichroic mirror 1677 transmits the light from the light source 1601, and reflects the light from the light source 1602 depending on the wavelength of the light. The SLO measuring light 1606 reflected by the dichroic mirror 1677 passes through a common optical path shared with the OCT measuring light 1604 to enter the dichroic mirror 1673. In this case, the common optical path for the SLO measuring light 1606 and the OCT measuring light 1604 includes the dichroic mirror 1677, the beam splitter 1671, the mirrors 1613 and 1614, the deformable mirror 1682, the mirrors 1615 and 1616, and the dichroic mirror 1673.

The dichroic mirrors 1673 and 1674 reflect the light from the light source 1601 and transmit the light from the light source 1602 depending on the wavelength of the light. Therefore, the SLO measuring light 1606 reflected by the mirror 1616 is transmitted through the dichroic mirror 1673 to enter an X scanner 1631 (third scanning unit). The center of the SLO measuring light 1606 is adjusted so as to match the rotation center of the X scanner 1631, and through the rotation of the X scanner 1631, it is possible to scan the SLO measuring light 1606 on the retina Er in the direction perpendicular to the optical axis. Although not shown, the X scanner 1631 is connected to the control unit 1690 so as to be controlled by the control unit 1690. The X scanner 1631 and the Y scanner 1633 form an SLO scanning unit configured to scan the SLO measuring light 1606 on the fundus of the eye E to be inspected in a two-dimensional direction.

The third embodiment is configured so that the optical path for the OCT measuring light 1604 and the optical path for the SLO measuring light 1606 are branched by the dichroic mirror 1673, and that the X scanner 1632 of the OCT measuring light 1604 and the X scanner 1631 of the SLO measuring light 1606 are separately arranged. A scanning speed of the OCT measuring light 1604 is limited by a reading speed of the line camera 1691. Meanwhile, the X scanner 1632 of the OCT measuring light 1604 and the X scanner 1631 of the SLO measuring light 1606 are separately provided, to thereby be able to increase a scanning speed of the SLO measuring light 1606. With this configuration, a frame rate of acquiring a fundus planar image through use of the SLO optical system can be increased. In the third embodiment, a resonance mirror is used as the X scanner 1631, but any deflecting mirror may be used depending on a desired configuration.

The SLO measuring light 1606 reflected by the X scanner 1631 is transmitted through the dichroic mirror 1674, and again passes through the common optical path shared with the OCT measuring light 1604 to enter the eye E to be inspected. In this case, the common optical path for the SLO measuring light 1606 and the OCT measuring light 1604 includes the dichroic mirror 1674, the mirrors 1617 to 1622, the Y scanner 1633, the mirror 1623, and the dichroic mirrors 1675 and 1676. To summarize the common optical path for the SLO measuring light 1606 and the OCT measuring light 1604, this common optical path includes an optical path extending from the dichroic mirror 1677 to the dichroic mirror 1673 and an optical path extending from the dichroic mirror 1674 to the dichroic mirror 1676.

The SLO measuring light 1606, which has entered the eye E to be inspected, is reflected or scattered by the retina Er, returns as the SLO return light 1607 along the optical path for the SLO measuring light 1606 to be reflected by the dichroic mirror 1677, and is then transmitted through the beam splitter 1672. The SLO return light 1607 transmitted through the beam splitter 1672 is condensed by a lens 1656 to pass through a pinhole plate 1678. A pinhole position of the pinhole plate 1678 is previously adjusted to a position conjugate with the fundus, and the pinhole plate 1678 acts as a confocal diaphragm configured to block undesired light from a point other than a conjugate point.

The SLO return light 1607 that has passed through the pinhole plate 1678 is received by a light-receiving element 1692. In the third embodiment, an avalanche photo diode (APD) is used as the light-receiving element 1692, but any other light-receiving element may be used depending on a desired configuration. The light-receiving element 1692 converts the received light into a voltage signal corresponding to a light intensity. The obtained voltage signal group is converted into a digital value. The control unit 1690 can perform data processing on the output signal from the light-receiving element 1692, which has been converted into a digital value, to generate a fundus planar image. The control unit 1690 also displays the generated fundus planar image on a display unit (not shown). The data processing to be performed when a fundus planar image is generated may be any known data processing for generating a fundus planar image from an output signal from the light-receiving element 1692.

Next, the fixation lamp optical system is described. The fixation lamp optical system includes the dichroic mirror 1675 and a fixation lamp panel 1694.

The dichroic mirror 1675 reflects visible light of the fixation lamp panel 1694 and transmits the light from the light source 1601 and the light source 1602 depending on the wavelength of the light. Thus, a pattern displayed on the fixation lamp panel 1694 is projected onto the retina Er of the eye E to be inspected via the dichroic mirror 1675. Through displaying of a desired pattern on the fixation lamp panel 1694, it is possible to designate a fixation direction of the eye E to be inspected, and to set a range of the retina Er to be imaged. In the third embodiment, an organic EL panel is used as the fixation lamp panel 1694, but another display may be used. The fixation lamp panel 1694 is connected to the control unit 1690 so as to be controlled by the control unit 1690.

Next, the anterior ocular observation optical system is described. The anterior ocular observation optical system includes the dichroic mirror 1676, an anterior ocular observation camera 1693, and an anterior ocular illumination light source (not shown).

The dichroic mirror 1676 reflects infrared light from the anterior ocular illumination light source and transmits the visible light of the fixation lamp panel 1694 and the light from the light source 1601 and the light source 1602 depending on the wavelength of the light. The optical axis of the anterior ocular observation camera 1693 is adjusted so as to match the optical axes of the OCT optical system and the SLO optical system. Therefore, it is possible to align the OCT optical system and the SLO optical system in the X direction and the Y direction with respect to the eye E to be inspected by adjusting an image of the anterior ocular segment of the eye E to be inspected based on the output from the anterior ocular observation camera 1693 at a reference position through observation on the display unit. The anterior ocular observation camera 1693 is connected to the control unit 1690 so as to be controlled by the control unit 1690.

In addition, the focus of the anterior ocular observation camera 1693 is adjusted so as to bring an iris of the eye E to be inspected into focus when matching a working distance of the OCT optical system and the SLO optical system (working distance in the Z direction). Therefore, it is possible to perform the alignment of the OCT optical system and the SLO optical system in the Z direction by bringing the iris in the image of the anterior ocular segment into focus through observation on the display unit. In the third embodiment, an LED having a wavelength of 970 nm is used as the anterior ocular illumination light source, and a CCD camera is used as the anterior ocular observation camera 1693. However, the anterior ocular illumination light source and the anterior ocular observation camera are not limited thereto, and it is also possible to use, for example, other light sources or other imaging elements. In addition, the wavelength of the anterior ocular illumination light source is not limited thereto, and may be changed in accordance with a desired configuration.

(Relationship Between Photographing Ranges)

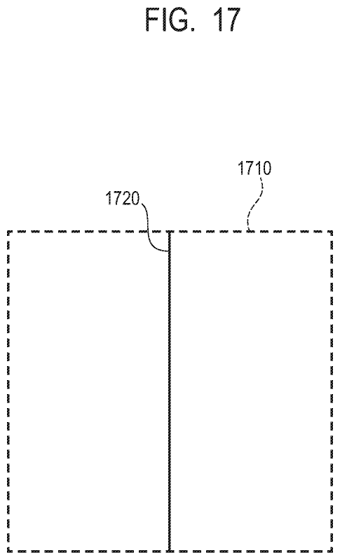

Next, a relationship between photographing ranges of the OCT optical system and the SLO optical system in the third embodiment is described with reference to FIG. 17. In FIG. 17, the solid line indicates a photographing range 1720 of the OCT optical system, and a photographing range 1710 of the SLO optical system is illustrated in a frame indicated by the broken line. FIG. 17 is a schematic illustration of a relationship between the photographing range 1720 of the OCT optical system and the photographing range 1710 of the SLO optical system, which is exhibited when the OCT optical system performs photographing by one line.

The OCT optical system and the SLO optical system have the Y scanner 1633 arranged on the common optical path, and hence scanning is simultaneously performed in the Y direction (up-down direction on the drawing sheet surface of FIG. 17). Meanwhile, in regard to the X scanner, the X scanner 1632 and the X scanner 1631 are separately used, and hence the photographing ranges of the OCT optical system and the SLO optical system in the X direction (left-right direction of FIG. 17) can be set independently of each other. For example, in FIG. 17, the photographing range 1720 of the OCT optical system is set at substantially the center of the photographing range 1710 of the SLO optical system, but the relationship between the photographing ranges in the X direction is not limited thereto. The photographing range 1720 of the OCT optical system may be freely set irrespective of the photographing range 1710 of the SLO optical system.

In addition, a scanning speed of the resonance mirror of the X scanner 1631 is higher than that of a galvanometer mirror, and hence the SLO measuring light 1606 is scanned a plurality of times in the X direction during one scan in the Y direction. Therefore, for example, the photographing range (one line) of the OCT optical system having a length L can be photographed through sampling (A-scan) at “m” points, and an L×L SLO photographing range can be photographed by “m” X scans. Thus, an L×L fundus front image (two-dimensional image) can be acquired by the SLO optical system while a one-line tomographic image having the length L is photographed by the OCT optical system. The numerical values of L and “m” may be freely set depending on a desired configuration.

When a 3D volume image is photographed, in addition to the scanning performed by the Y scanner 1633, the OCT measuring light 1604 is scanned by the X scanner 1632 to repeat the above-mentioned photographing of one line in the Y direction while changing a scanning position of the X scanner 1632. For example, an L×L 3D volume image can be acquired by photographing “m” lines within the range of L in the X direction. During this period, “m” L×L fundus front images (two-dimensional images) can be acquired through use of the SLO optical system.

(Procedure for Tracking)

Next, a method of correcting a positional deviation (tracking) based on a fundus front image (two-dimensional image) acquired through use of the SLO optical system is described.

In tracking processing in the third embodiment, the control unit 1690 sets, as a reference image, a fundus front image based on fundus information (first fundus information) on the eye E to be inspected, which is acquired through use of the SLO optical system when photographing is performed for the first time out of a plurality of times that a line tomographic image is acquired at the same position through use of the OCT optical system. Subsequently, the control unit 1690 sets, as a target image for detection of a positional deviation, a fundus image based on fundus information (second fundus information), which is acquired through use of the SLO optical system when photographing is performed for the second and subsequent times through use of the OCT optical system. The control unit 1690 calculates a positional deviation amount of the target image with respect to the reference image. The positional deviation amount can be calculated by pattern matching or other such image processing.

The control unit 1690 controls the X scanner 1632 and the Y scanner 1633 so as to correct the calculated positional deviation amount. This enables the control unit 1690 to perform fundus tracking for correcting a deviation of the photographing position of the tomographic image based on the movement of the fundus due to, for example, the involuntary eye movement during fixation. A plurality of line tomographic images acquired at the same position can be used for performing, for example, processing for reducing noise in tomographic images due to their superimposition.

The above-mentioned fundus tracking can be similarly applied to the case of acquiring a 3D volume image through use of the OCT optical system. In this case, as described above, a one-line tomographic image in the Y direction is repeatedly acquired while changing the position in the X direction through use of the OCT optical system. At this time, the control unit 1690 sets, as the reference image, a fundus front image based on the fundus information on the eye E to be inspected, which is acquired when the photographing is performed for the first time (on the first line). Then, the control unit 1690 sets, as the target image, a fundus front image based on the fundus information acquired when the photographing is performed for the second and subsequent times (on the second and subsequent lines). The control unit 1690 calculates a positional deviation amount between the reference image and the target image to perform the fundus tracking. Therefore, even when acquiring a 3D volume image, it is possible to correct a deviation of the photographing position of the tomographic image with respect to the retina Er of the eye E to be inspected.

As the reference image, when the photographing is performed for the first time through use of the OCT optical system, the entirety of the fundus front image acquired through use of the SLO optical system may be used, or a partial image of the fundus front image may be used. In the same manner, as the target image, the entirety of the fundus front image acquired through use of the SLO optical system may be used, or a partial image of the fundus front image may be used. When a partial image of the fundus front image is used as the target image, an acquisition interval for the target image can be shortened, and a control rate of fundus tracking can be increased. This facilitates the correction of a positional deviation due to a faster movement of the fundus.

In another case, the image size of the reference image may be set larger than the image size of the target image. In this case, while maintaining the control rate by keeping the image size of the target image small, it becomes easier to secure a large overlapping region between the reference image and the target image even when the positional deviation amount between the reference image and the target image is large, and it becomes easier to correct a positional deviation due to a large movement of the fundus. When the fundus is moving so slow that the control rate of the fundus tracking has a margin, the image size of the target image may be set to be larger than the image size of the reference image. Even in this case, it is easy to correct a positional deviation due to a large movement of the fundus. In other words, when the image size of one of the reference image and the target image is set larger than the image size of the other one, it becomes easier to correct a positional deviation due to a large movement of the fundus.

(Fundus Photographing Procedure)

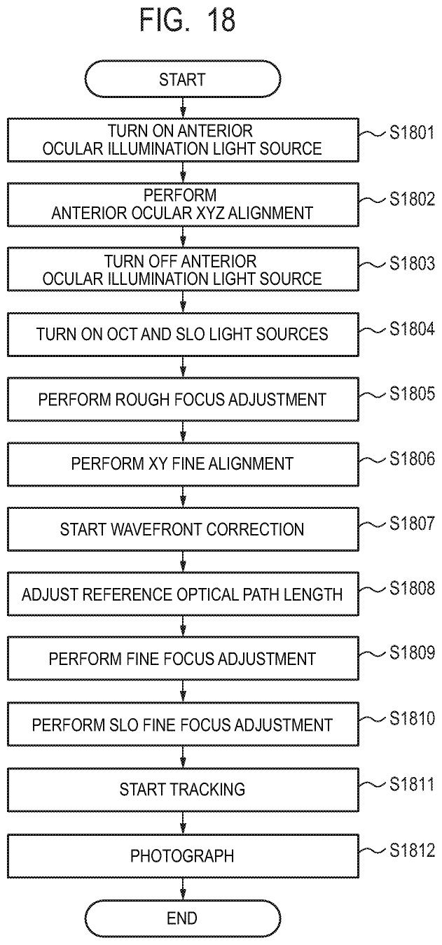

Next, a fundus photographing procedure for the fundus imaging apparatus 1600 is described with reference to FIG. 18. FIG. 18 is a flow chart of the fundus photographing procedure in the third embodiment.

First, when an inspector presses an anterior ocular illumination light source button (not shown) displayed on the display unit, in Step S1801, the control unit 1690 turns on an anterior ocular illumination light source (not shown). When the anterior ocular illumination light source is turned on, the control unit 1690 generates an image of an anterior ocular segment of the eye E to be inspected based on output from the anterior ocular observation camera 1693 to display the image on the display unit.

In Step S1802, the control unit 1690 performs alignment in the X, Y, and Z directions (anterior ocular XYZ alignment) on the imaging unit including the OCT optical system and the SLO optical system with respect to the eye E to be inspected based on the image of the anterior ocular segment displayed on the display unit. Specifically, the inspector observes the image of the anterior ocular segment, and the control unit 1690 controls a driving mechanism (not shown) of the imaging unit in response to input from the inspector to perform the alignment of the imaging unit with respect to the eye E to be inspected. As described above, the positions of the anterior ocular observation camera 1693 in the X, Y, and Z directions are adjusted with respect to the OCT optical system and the SLO optical system. Therefore, the inspector adjusts the positions of the imaging unit in the X, Y, and Z directions so as to bring the image of the anterior ocular segment displayed on the display unit into focus (Z position) with the aligned X and Y positions, to thereby be able to perform the alignment of the OCT optical system and the SLO optical system in the X, Y, and Z directions. The inspector may operate a driving mechanism (not shown) of the imaging unit to perform the alignment of the imaging unit.

When the anterior ocular XYZ alignment is completed, in Step S1803, the control unit 1690 turns off the anterior ocular illumination light source in response to the inspector again pressing the anterior ocular illumination light source button displayed on the display unit.

When the anterior ocular illumination light source is turned off, in Step S1804, the control unit 1690 turns on the light source 1601 of the OCT optical system and the light source 1602 of the SLO optical system in response to the inspector pressing a light source button (not shown) displayed on the display unit. A timing to turn on the light source 1601 of the OCT optical system is not limited thereto. For example, the light source 1601 may be turned on after rough focus adjustment is performed in Step S1805, which is described later.

When the light source 1602 of the SLO optical system is turned on, the control unit 1690 generates a fundus planar image based on output from the light-receiving element 1692 to display the fundus planar image on the display unit. In Step S1805, the control unit 1690 performs approximate focus adjustment (rough focus adjustment) on the SLO optical system and the OCT optical system in response to input from the inspector based on the fundus planar image displayed on the display unit.

Specifically, the control unit 1690 moves the electric stage 1626 in response to the inspector moving a focus adjustment bar (not shown) displayed on the display unit while observing the fundus planar image. The electric stage 1626 and the mirrors 1619 and 1620 are arranged on the common optical path for the OCT measuring light 1604 and the SLO measuring light 1606, and when the focus adjustment is performed on the SLO measuring light 1606, the rough focus adjustment is simultaneously performed on the OCT measuring light 1604 as well. In this case, the focus adjustment is performed so as to maximize the luminance of the fundus planar image.

At this time, the control unit 1690 causes the electric stage 1627 included in the SLO optical system to be arranged at a position in a preset initial state. In this case, as the position of the electric stage 1627 in the initial state, the electric stage 1627 is set at such a position that the focus positions of the OCT measuring light 1604 and the SLO measuring light 1606 substantially match each other.

After performing the rough focus adjustment, in Step S1806, the control unit 1690 performs XY fine alignment on the imaging unit with respect to the eye E to be inspected in response to input from the inspector based on a position of a Hartmann image output from the wavefront sensor 1681 and displayed on the display unit. In the XY fine alignment, the inspector observes the position of the Hartmann image output from the wavefront sensor 1681 and displayed on the display unit, and the control unit 1690 performs minute alignment on the imaging unit in the X direction and the Y direction with respect to the eye E to be inspected in response to input from the inspector.

At this point, the wavefront sensor 1681 is adjusted so that the center position of the wavefront sensor 1681 matches the optical axes of the OCT optical system and the SLO optical system. Therefore, the inspector adjusts the position of the imaging unit with respect to the eye E to be inspected so that the Hartmann image matches the center of the wavefront sensor 1681, to thereby be able to perform the alignment of the OCT optical system and the SLO optical system in the X direction and the Y direction. On the display unit, an indicator or the like corresponding to the center position of the wavefront sensor 1681 and the Hartmann image may be displayed.

After performing the XY fine alignment, in Step S1807, the control unit 1690 starts wavefront correction using the deformable mirror 1682 in response to the inspector pressing a wavefront correction button (not shown) displayed on the display unit. Then, the control unit 1690 deforms the shape of the deformable mirror 1682 based on aberration measured by the wavefront sensor 1681 to correct the aberration of the eye E to be inspected other than the defocus component. In this case, an aberration correction method using a deformable mirror may be performed by an existing method, and hence a description thereof is omitted.

The deformable mirror 1682 is arranged on the common optical path for the OCT measuring light 1604 and the SLO measuring light 1606. Therefore, when the shape of the deformable mirror 1682 is deformed to correct the aberration of the eye E to be inspected for the OCT measuring light 1604, the aberration of the eye E to be inspected for the SLO measuring light 1606 can also be corrected.

When the wavefront correction is started, in Step S1808, the control unit 1690 adjusts the optical path length of the reference light 1603. Specifically, the control unit 1690 controls the electric stage 1625 to adjust the optical path length of the reference light 1603 in response to the inspector moving a reference optical path length adjustment bar (not shown) displayed on the display unit. In this case, the control unit 1690 displays the tomographic image acquired through use of the OCT optical system on the display unit, and adjusts the optical path length of the reference light 1603 in response to input from the inspector so that an image of a desired layer in the tomographic image is adjusted at a desired position within the tomographic image display area.

After adjusting the optical path length of the reference light 1603, in Step S1809, the control unit 1690 performs the fine focus adjustment on the OCT optical system. Specifically, when the inspector moves the focus adjustment bar (not shown) displayed on the display unit based on the tomographic image, the control unit 1690 controls the electric stage 1626 to perform minute focus adjustment on the OCT optical system. In an optical system of adaptive optics OCT having a high lateral resolution, the NA of the measuring light at the fundus is large with a shallow depth of focus, and hence it is difficult to bring the entirety of the retina Er in the depth direction into focus simultaneously. Therefore, in Step S1809, fine focus adjustment is performed so that the OCT measuring light 1604 is focused on a layer of the retina Er to be particularly photographed. For example, when a blood vessel on a surface layer of the retina is to be photographed, the control unit 1690 controls the electric stage 1626 to adjust the focus of the OCT measuring light 1604 so as to maximize the luminance of a portion including the blood vessel.

After adjusting the OCT measuring light 1604 to a desired focus state by the fine focus adjustment, in Step S1810, the control unit 1690 performs SLO fine focus adjustment based on the fundus planar image acquired through use of the SLO optical system. Specifically, the control unit 1690 controls the electric stage 1627 in response to the inspector moving an SLO focus adjustment bar (not shown) displayed on the display unit. In this case, the focus adjustment is performed so as to increase a contrast of photoreceptor cells in the fundus planar image displayed on the display unit. A position to be brought into focus in the SLO optical system is not limited to the position of the photoreceptor cells. The position to be brought into focus in the SLO optical system may be a position of a blood vessel or another such position having another feature point as long as desired tracking accuracy can be achieved.

The focus lens 1657 mounted on the electric stage 1627 is arranged on a dedicated optical path of the SLO optical system, which is branched off from the common optical path shared with the OCT optical system. Therefore, through the changing of the position of the focus lens 1657 by the electric stage 1627, it is possible to adjust the focus of the SLO optical system without affecting the focus state of the OCT optical system.

The focus lens 1657 is also arranged on the common optical path for the SLO measuring light 1606 and the SLO return light 1607. Therefore, the focus position of the SLO measuring light 1606 can be adjusted to a desired position of the retina Er, and at the same time, the focus position of the SLO return light 1607 from that position can be adjusted to the pinhole position of the pinhole plate 1678.

The focus adjustment of the SLO optical system can also be performed by independently moving the lens 1655, which is arranged on a dedicated optical path for the SLO measuring light 1606, and the lens 1656, which is arranged on a dedicated optical path for the SLO return light 1607, in the optical axis direction. However, in that case, it is required to independently control the positions of the lens 1655 and the lens 1656, which complicates the apparatus configuration and control. Meanwhile, when the focus adjustment of the SLO optical system is performed through use of the focus lens 1657, the apparatus configuration and control can be simplified.

After performing the SLO fine focus adjustment, in Step S1811, the control unit 1690 starts the fundus tracking in response to the inspector pressing a tracking button (not shown) displayed on the display unit. In the above-mentioned manner, the control unit 1690, which functions as an eye movement detection unit, calculates a positional deviation amount from a feature point of the fundus planar image acquired through use of the SLO optical system, and controls the X scanner 1632 and the Y scanner 1633 based on the calculated deviation amount, to thereby perform the fundus tracking. Thus, the fundus imaging apparatus 1600 can acquire a plurality of tomographic images, a moving image, a 3D volume image, or another such image to be used for processing for noise due to the superimposition of tomographic images, with a positional deviation suppressed to a low level.

When the tracking is started, in Step S1812, the control unit 1690 acquires a fundus tomographic image and a fundus planar image in response to the inspector pressing a photographing button (not shown) displayed on the display unit. Interference light (light 1608) between the OCT measuring light 1604 and the reference light 1603 is received by the line camera 1691 to be converted into a voltage signal. The obtained voltage signal group is further converted into a digital value, and the control unit 1690 stores and processes data. The control unit 1690 processes the data based on the interference light, to thereby generate a fundus tomographic image. Further, the SLO return light 1607 is received by the light-receiving element 1692 to be converted into a voltage signal. The obtained voltage signal group is further converted into a digital value, and the control unit 1690 stores and processes data. The control unit 1690 processes the data based on the SLO return light 1607, to thereby generate a fundus planar image.

In the third embodiment, while using the fundus planar image, which has been acquired through use of an optical system of adaptive optics SLO, to perform accurate fundus tracking, it is possible to use the optical system of adaptive optics OCT to bring a desired layer of the retina Er into focus and photograph a fundus tomographic image having a high resolution and a satisfactory S/N ratio. In addition, aberration variations due to the movement of the focus lens 1657 in the SLO optical system do not affect the OCT optical system, and hence it is possible to acquire a fundus tomographic image through use of the OCT optical system while performing the aberration correction with high accuracy.

As described above, the fundus imaging apparatus 1600 according to the third embodiment includes the OCT optical system configured to acquire tomographic information on the eye E to be inspected through use of the OCT measuring light 1604 and the SLO optical system configured to acquire the fundus information on the eye E to be inspected through use of the SLO measuring light 1606. In addition, the fundus imaging apparatus 1600 includes the common optical path to be used for the OCT optical system and the SLO optical system to share at least a part of the optical path for the OCT measuring light 1604 or the optical path for the SLO measuring light 1606, and the Badal optical system formed of the mirrors 1619 and 1620 that are provided on the common optical path. The fundus imaging apparatus 1600 further includes the focus lens 1657 provided on the optical path for the SLO measuring light 1606, which is branched off from the common optical path. In this case, the focus adjustment range of the focus lens 1657 is narrower than the focus adjustment range of the Badal optical system.

The fundus imaging apparatus 1600 also includes the control unit 1690 configured to control the Badal optical system and the focus lens 1657, the wavefront sensor 1681 configured to measure the aberration of the return light of the OCT measuring light 1604, and the deformable mirror 1682 provided on the common optical path and configured to correct the aberration. The control unit 1690 controls a change in shape of the deformable mirror based on the aberration measured by the wavefront sensor 1681.

The fundus imaging apparatus 1600 further includes the X scanner 1632 and the Y scanner 1633, which are configured to scan the OCT measuring light 1604 on the fundus of the eye to be inspected in the two-dimensional direction. The control unit 1690 detects the movement of the fundus based on the fundus information on the eye E to be inspected, which has been acquired through use of the SLO optical system, to control the X scanner 1632 and the Y scanner 1633 based on the detected movement of the fundus.

With such a configuration, the fundus imaging apparatus 1600 can adjust the focus positions of the OCT optical system and the SLO optical system to mutually different positions while having a compact apparatus configuration. This enables the fundus imaging apparatus 1600 to adjust the focus position of the optical system of adaptive optics OCT to a layer to be photographed, and to adjust the focus position of the optical system of adaptive optics SLO to a layer having a large number of feature points advantageous for position detection for fundus tracking. Thus, it is possible to photograph a tomographic image having a high lateral resolution by the optical system of adaptive optics OCT while performing highly accurate fundus tracking through use of the optical system of adaptive optics SLO. Therefore, it is possible to acquire a plurality of tomographic images, a moving image, or a 3D volume image while suppressing the positional deviation during the photographing to a smaller level.

The fundus imaging apparatus 1600 according to the third embodiment also includes the Y scanner 1633 configured to scan the OCT measuring light 1604 and the SLO measuring light 1606 in the Y direction (first scanning direction) and the X scanner 1632 configured to scan the OCT measuring light 1604 in the X direction (second scanning direction) perpendicular to the Y direction. The fundus imaging apparatus 1600 further includes the X scanner 1631 configured to scan the SLO measuring light 1606 in the X direction. In this case, the control unit 1690 causes the Y scanner 1633 to repeatedly scan the OCT measuring light 1604 and the SLO measuring light 1606 while performing one scan by the X scanner 1632. The control unit 1690 also causes the X scanner 1631 to repeatedly scan the SLO measuring light 1606 while performing one scan by the Y scanner 1633.

In addition, the common optical path of the OCT optical system and the SLO optical system includes the dichroic mirror 1673 (first dichroic mirror) configured to separate the OCT measuring light 1604 and the SLO measuring light 1606. The common optical path further includes the dichroic mirror 1674 (second dichroic mirror) configured to combine the OCT measuring light 1604 and the SLO measuring light 1606 that have been separated by the dichroic mirror 1673. In this case, the X scanner 1632 is arranged on the optical path for the OCT measuring light 1604 separated by the dichroic mirror 1673, and the X scanner 1631 is arranged on the optical path for the SLO measuring light 1606 separated in the same manner.

With such a configuration, in the fundus imaging apparatus 1600, the Y scanner is shared by the OCT optical system and the SLO optical system, and hence the apparatus configuration of the fundus imaging apparatus 1600 can be set more compact than when separate Y scanners are provided to the respective optical systems. In addition, the fundus imaging apparatus 1600 uses the X scanner 1632 and the X scanner 1631 separately, and hence the photographing ranges of the OCT optical system and the SLO optical system in the X direction can be set independently. Further, the fundus imaging apparatus 1600 can rotate the X scanners 1631 and 1632 in the SLO optical system and the OCT optical system at different cycle periods, and can set the scanning speed of the measuring light in the SLO optical system faster than the scanning speed of the measuring light in the OCT optical system.

### Fourth Embodiment

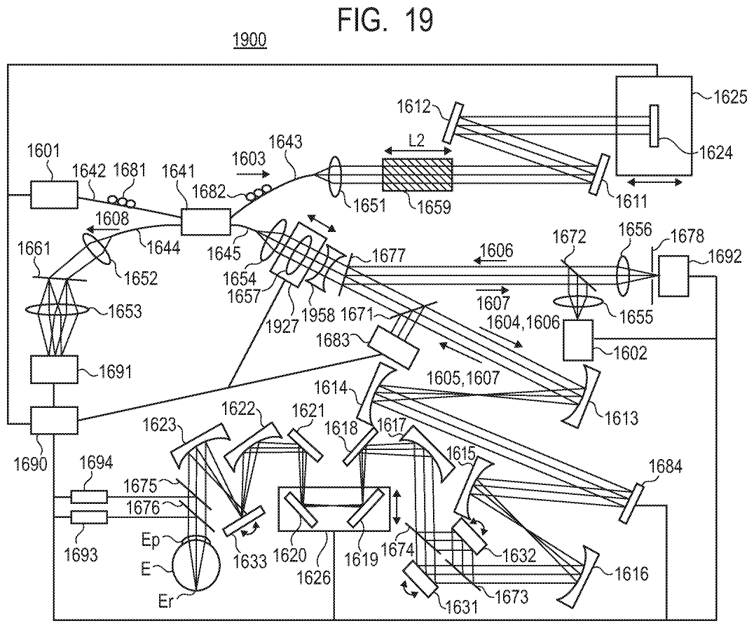

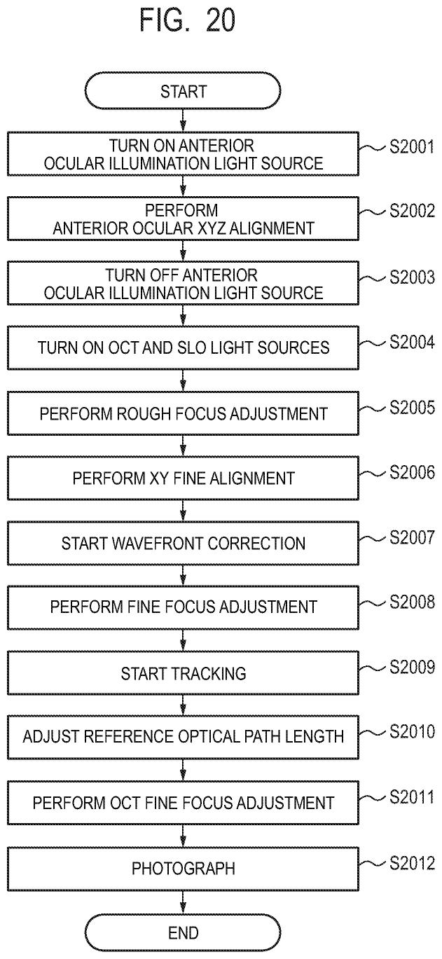

Referring to FIG. 19 and FIG. 20, a fundus imaging apparatus 1900 according to a fourth embodiment of the present invention is described.

(Apparatus Configuration)

Now, referring to FIG. 19, the fundus imaging apparatus 1900 according to the fourth embodiment is described by focusing on differences from the fundus imaging apparatus 1600 according to the first embodiment. FIG. 19 is an illustration of a schematic configuration of the fundus imaging apparatus 1900 according to the fourth embodiment. In addition, the same components as those of the fundus imaging apparatus 1600 according to the first embodiment are denoted by the same reference symbols, and descriptions thereof are omitted.

The basic configuration of the fundus imaging apparatus 1900 is the same as that of the fundus imaging apparatus 1600 according to the first embodiment. However, the fundus imaging apparatus 1900 is different from the fundus imaging apparatus 1600 in that the second focus unit is arranged on the dedicated optical path of the OCT optical system instead of being arranged on the dedicated optical path of the SLO optical system. In the fundus imaging apparatus 1900, a focus lens 1957 is arranged as the second focus unit on the dedicated optical path of the OCT optical system, which is branched off from the common optical path of the OCT optical system and the SLO optical system.

In the fourth embodiment, the focus lens 1957 and a lens 1958 are provided between the lens 1654 and the dichroic mirror 1677 on the optical path for the OCT measuring light 1604. The focus lens 1957 is mounted on an electric stage 1927. The electric stage 1927 can be moved in an optical axis direction of the OCT measuring light 1604 as indicated by the arrow in FIG. 19 under the control of the control unit 1690.

In FIG. 19, the focus lens 1957 is illustrated as a convex lens, and the lens 1958 is illustrated as a concave lens, but the configurations of the focus lens 1957 and the lens 1958 are not limited thereto. The focus lens 1957 and the lens 1958 may be configured as a concave lens and a convex lens, respectively, or may be both configured as convex lenses so as to form an intermediate image therebetween.

(Fundus Photographing Procedure)

Next, a fundus photographing procedure for the fundus imaging apparatus 1900 according to the fourth embodiment is described with reference to FIG. 20. FIG. 20 is a flow chart of the fundus photographing procedure in the fourth embodiment. Step S2001 to Step S2007 are the same as Step S1801 to Step S1807 in the photographing procedure in the third embodiment, and hence descriptions thereof are omitted.

After the photographing is started, the alignment, the rough focus adjustment, and the start of the wavefront correction are performed in Step S2001 to Step S2007 in the same manner as in Step S1801 to Step S1807 in the third embodiment. Then, the processing advances to Step S2008.

In Step S2008, the control unit 1690 performs the fine focus adjustment on the SLO optical system. Specifically, when the inspector moves the focus adjustment bar (not shown) displayed on the display unit based on the fundus planar image, the control unit 1690 controls the electric stage 1626 to perform minute focus adjustment on the SLO optical system. In Step S2008, the focus adjustment is performed so as to increase a contrast of photoreceptor cells in the fundus planar image displayed on the display unit. A position to be brought into focus in the SLO optical system is not limited to the position of the photoreceptor cells. The position to be brought into focus in the SLO optical system may be a position of a blood vessel or another such position having another feature point as long as desired tracking accuracy can be achieved.

At this time, the control unit 1690 causes the electric stage 1927 included in the OCT optical system to be arranged at a position in a preset initial state. In this case, as the position of the electric stage 1927 in the initial state, the electric stage 1927 is set at such a position that the focus positions of the OCT measuring light 1604 and the SLO measuring light 1606 substantially match each other.

After adjusting the contrast of the photoreceptor cells so as to be maximized by the fine focus adjustment, the control unit 1690 starts the fundus tracking in Step S2009 in the same manner as in Step S1811 in the third embodiment. After that, in Step S2010, the control unit 1690 adjusts the reference optical path length in the same manner as in Step S1808 in the third embodiment.

After adjusting the reference optical path length, in Step S2011, the control unit 1690 performs OCT fine focus adjustment. Specifically, in response to the inspector moving an OCT focus adjustment bar (not shown) displayed on the display unit based on the tomographic image, the control unit 1690 controls the electric stage 1927 to move the focus lens 1957, and performs the minute focus adjustment on the OCT optical system. In this case, the focus adjustment is performed so as to maximize the luminance of the layer to be photographed in the tomographic image displayed on the display unit.

In the fourth embodiment, the focus lens 1957 is arranged on a dedicated optical path of the OCT optical system, which is branched off from the common optical path shared with the SLO optical system. Therefore, through the changing of the position of the focus lens 1957 by the electric stage 1927, it is possible to adjust the focus of the OCT optical system without affecting the focus state of the SLO optical system.

When the luminance of a desired layer is maximized by the OCT fine focus adjustment, in Step S2012, the photographing is performed in the same procedural step as Step S1812 in the third embodiment.

As described above, the fundus imaging apparatus 1900 according to the fourth embodiment includes the focus lens 1957 being the second focus unit on the optical path for the OCT measuring light 1604 in the OCT optical system, which is branched off from the common optical path. Even with such a configuration, the fundus imaging apparatus 1900 can adjust the focus positions of the OCT optical system and the SLO optical system to mutually different positions while having a compact apparatus configuration.

Therefore, in the same manner as the fundus imaging apparatus 1600 according to the third embodiment, the fundus imaging apparatus 1900 according to the fourth embodiment can use the OCT optical system to bring a desired layer into focus and photograph a fundus tomographic image having a high resolution while performing accurate fundus tracking. In addition, in the fundus imaging apparatus 1900, it is possible to change the focus of the OCT optical system to a different layer while performing the fundus tracking through use of the SLO optical system, which facilitates the operation when, for example, tomographic images are photographed at a plurality of focus positions of the OCT optical system.

### Modification Example 1

In the third and fourth embodiments, the mirrors 1619 and 1620 mounted on the electric stage 1626 arranged on the common optical path of the OCT optical system and the SLO optical system are used as the first focus unit, and the rough focus adjustment and the fine focus adjustment are performed by moving the mirrors 1619 and 1620. However, the first focus unit to be used for those focus adjustments is not limited thereto. For example, the deformable mirror 1682 arranged on the common optical path of the OCT optical system and the SLO optical system can be used as the first focus unit.

In particular, the fine focus adjustment may be performed by deforming the deformable mirror 1682. In this case, the control unit 1690 performs control by applying an offset of the defocus component to a target shape of the deformable mirror 1682 based on a measured value of the wavefront sensor 1681. Thus, it is possible to change the focus positions of the OCT optical system and the SLO optical system while correcting the aberration of the eye E to be inspected. In the rough focus adjustment, the deformable mirror 1682 can be used similarly when an amount of the focus adjustment is small.

As the first focus unit, any other focus unit such as a focus lens, an electro-optical element, a piezo element, a liquid crystal optical element, or a deformable mirror may be used.

### Modification Example 2

In the third and fourth embodiments, the focus adjustment is performed by independently controlling the first focus unit and the second focus unit, but the fundus imaging apparatus may also have a mode of controlling the first focus unit and the second focus unit in linkage with each other.

The apparatus configuration for this case is the same as that of the fundus imaging apparatus 1600 illustrated in FIG. 16, and the fundus photographing procedure for this case is the same as that of the flow chart illustrated in FIG. 20. However, there is a difference in that the first focus unit and the second focus unit are controlled in linkage with each other during the OCT fine focus adjustment (Step S2011).

In this case, in Step S2008, the control unit 1690 controls the electric stage 1626 to move the focus lens 1657 to perform the fine focus adjustment of the SLO optical system. After that, in Step S2011, when moving the electric stage 1626 to perform the OCT fine focus adjustment, the control unit 1690 controls the electric stage 1627, which is arranged on the dedicated optical path of the SLO optical system, to operate in such a direction as to cancel the adjustment performed by the electric stage 1626. In other words, when performing the OCT fine focus adjustment through use of the first focus unit arranged on the common optical path, the control unit 1690 operates the second focus unit arranged on the dedicated optical path in such a direction as to cancel an influence of the adjustment in linkage with the first focus unit. Therefore, it is possible to perform the focus adjustment on the OCT optical system without changing the focus state of the SLO optical system.

After that, when the luminance of a desired layer is maximized by the OCT fine focus adjustment, in Step S2012, the control unit 1690 performs photographing in the same procedural step as Step S1812 in the third embodiment.

Even in this case, while performing accurate fundus tracking, it is possible to use the optical system of adaptive optics OCT to bring a desired layer into focus and photograph an image having a high resolution and a satisfactory S/N ratio. In addition, it is possible to change the focus of the optical system of adaptive optics OCT to a different layer while performing the fundus tracking through use of the optical system of adaptive optics SLO, which facilitates the operation when, for example, the photographing is performed at a plurality of focus positions. Further, aberration variations due to the movement of the focus lens 1657 mounted on the electric stage 1627 do not affect the OCT optical system, and hence it is possible to acquire a fundus tomographic image through use of the optical system of adaptive optics OCT while performing the aberration correction with high accuracy. With the configuration of the fundus imaging apparatus 1900, it is also possible to perform the same processing when performing the fundus photographing procedure illustrated in the flow chart of FIG. 18.

The focus adjustment may be performed by deforming the deformable mirror 1682 as the first focus unit. In this case, the control may be performed by applying an offset of the defocus component to a target shape of the deformable mirror 1682 to control the electric stage 1627 being the second focus unit so as to cancel the offset amount.

It is also possible to provide a linkage mechanism for linking the first focus unit and the second focus unit to each other. In this case, the control unit 1690 can link the first focus unit and the second focus unit to each other by controlling the linkage mechanism. In addition, the linkage mechanism may be configured to be able to remove the linkage between the first focus unit and the second focus unit. In this case, the control unit 1690 can remove the linkage established by the linkage mechanism to separately control the first focus unit and the second focus unit.

### Modification Example 3

When the rough focus adjustment is performed in Step S1805 in the third embodiment and in Step S2005 in the second embodiment, the adjustment of the optical path length adjusting unit provided on the reference optical path and the focus adjustment by the first focus unit may be controlled by being linked to each other.

In this case, the control unit 1690 controls the electric stage 1625 to move the mirror 1624 so as to change the optical path length by substantially the same amount as a change amount of the optical path length due to the movement of the mirrors 1619 and 1620 during the rough focus adjustment. Thus, it is possible to adjust the focus without changing a difference in optical path length between the OCT measuring light 1604 and the reference light 1603. Therefore, the movement amount of the mirror 1624 exhibited when the reference optical path length is adjusted in Step S1808 in the third embodiment and in Step S2010 in the fourth embodiment can be suppressed to a low level. When the movement amount of the mirror 1624 mounted on the electric stage 1625 is small, the adjustment time of the optical path length can be shortened, and a total photographing time from the start of the operation to the completion of the photographing can be shortened. Thus, it is possible to reduce a burden on a subject to be inspected.

It is also possible to provide a linkage mechanism for linking the optical path length adjusting unit and the first focus unit to each other. In this case, the control unit 1690 can link the mirror 1624 mounted on the electric stage 1625 to the first focus unit by controlling the linkage mechanism. In addition, the linkage mechanism may be configured to be able to remove the linkage between the optical path length adjusting unit and the first focus unit. In this case, the control unit 1690 can remove the linkage established by the linkage mechanism to separately control the optical path length adjusting unit and the first focus unit.

In the third and fourth embodiments, the optical path length adjusting unit is formed of the mirror 1624 provided on the optical path for the reference light 1603. However, the optical path length adjusting unit may be provided on the optical path for the OCT measuring light 1604.

### Modification Example 4

In the third and fourth embodiments, the second focus unit is provided on one of the dedicated optical path of the OCT optical system and the dedicated optical path of the SLO optical system. However, the second focus unit may be provided on both the dedicated optical path of the OCT optical system and the dedicated optical path of the SLO optical system. As described above, the second focus unit is used for the fine focus adjustment of each optical system due to the focus adjustment range narrower than that of the first focus unit and the narrower diopter range of the eye E to be inspected that can be handled. In Modification Example 4, in the imaging procedure, the fine focus after the rough focus is separately performed by the second focus unit provided on the dedicated optical path of the OCT optical system and the second focus unit provided on the dedicated optical path of the SLO optical system.

Even in such a case, the focus adjustment range of the second focus unit is narrower than that of the first focus unit, and hence, for example, it is possible to narrow the moving range of the electric stage on which the focus lens is mounted, and to set the apparatus configuration more compact than a related-art apparatus. In addition, the second focus unit is not limited to the electric stage on which the focus lens is mounted. For example, the second focus unit may be configured by an electro-optical element made of, for example, a crystal of potassium tantalate niobate, or another piezo element, liquid crystal optical element, or deformable mirror that can produce the same effect. In this case, it is not required to secure the moving range of the electric stage, and hence the apparatus configuration can be set more compact.

According to the embodiments and modification examples described above, it is possible to simultaneously acquire an AO-OCT image and an AO-SLO image without complicating the apparatus configuration. Further, according to the embodiments and modification examples described above, it is possible to perform tracking through use of an AO-SLO image when acquiring an AO-OCT image.

In the third and fourth embodiments and the modification examples, the control unit 1690 performs various alignments, optical path length adjustments, and focus adjustments in response to input from the inspector. However, the control unit 1690 may automatically perform those alignments and adjustments based on the image of the anterior ocular segment, the fundus planar image, the Hartmann image, the tomographic image, and other such images, which are used for the above-mentioned various alignments, optical path length adjustments, and focus adjustments. In this case, for example, the control unit 1690 can perform those alignments and adjustments based on, for example, the luminance of the fundus planar image and the layer to be photographed, in the same manner as the above-mentioned alignments and adjustments.

In the embodiments and modification examples described above, a configuration of a Michelson interferometer is used as the interference optical system of the fundus imaging apparatus, but the configuration of the interference optical system is not limited thereto. For example, the interference optical system of the fundus imaging apparatus may have a configuration of a Mach-Zehnder interferometer. In addition, the wavelength of light to be reflected or transmitted by each dichroic mirror in the embodiments and modification examples described above is freely set, and a combination of light opposite to that of the above-mentioned configuration may be reflected or transmitted.

Further, in the third and fourth embodiments and the modification examples, the Y scanner 1633 is shared by the OCT optical system and the SLO optical system, but the OCT optical system and the SLO optical system may be provided with separate Y scanners. In addition, the first focus unit arranged on the common optical path is not limited to the configuration arranged between the Y scanner 1633 and the X scanners 1631 and 1632. For example, at least one of the X scanner 1631 or 1632 may be provided between the eye E to be inspected and the first focus unit. In addition, the Y scanner 1633 may not be provided between the eye E to be inspected and the first focus unit.

Further, in the embodiments and modification examples described above, a spectral domain OCT (SD-OCT) optical system using an SLD as a light source has been described as the OCT optical system, but the configuration of the OCT optical system in at least one embodiment of the present invention is not limited thereto. For example, the present invention can also be applied to any other types of OCT optical systems including a wavelength-swept OCT (SS-OCT) optical system using a wavelength-swept light source capable of sweeping the wavelength of emitted light.

The descriptions of the embodiments and modification examples described above are directed to the case in which the object to be inspected is an eye, but the present invention can also be applied to objects to be inspected other than the eye, which include skin and an organ. In this case, the present invention has a mode as medical equipment, for example, an endoscope, instead of the imaging apparatus. Therefore, it is preferred that the present invention be grasped as an image processing apparatus exemplified by the imaging apparatus, and that the eye to be inspected be grasped as one mode of the object to be inspected.

### OTHER EMBODIMENTS

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

What is claimed is:

1. An imaging apparatus comprising:
a first scanning unit arranged to scan light on a fundus in a first direction;
a second scanning unit arranged to scan the light on the fundus in a second direction being a direction different from the first direction;
an optical system arranged to join an optical path extending to the second scanning unit to an optical path extending from the second scanning unit without intermediation of the second scanning unit;
a common optical system arranged to:
illuminate the fundus via the first scanning unit and the optical system with first measuring light obtained by branching light emitted from a first light source; and
illuminate the fundus via the first scanning unit and the second scanning unit with second measuring light emitted from a second light source;
a first generating unit configured to generate a tomographic image of the fundus based on interference light obtained by causing return light from the fundus of the first measuring light illuminated by the common optical system via the first scanning unit and the optical system, and reference light obtained by branching the light emitted from the first light source, to interfere with each other; and
a second generating unit configured to generate a fundus image of the fundus based on return light from the fundus of the second measuring light illuminated by the common optical system via the first scanning unit and the second scanning unit,
wherein the first generating unit is configured to generate the tomographic image at a predetermined position in the fundus image generated by the second generating unit.

2. The imaging apparatus according to claim 1,
wherein the common optical system includes:
a wavefront sensor arranged to measure wavefront aberration; and
a wavefront correction apparatus configured to correct the wavefront aberration,
wherein the wavefront sensor is arranged to measure one of the wavefront aberration of the return light from the fundus of the first measuring light and the wavefront aberration of the return light from the fundus of the second measuring light, and
wherein the wavefront correction apparatus is configured to correct the wavefront aberration of the return light from the fundus of the first measuring light and the wavefront aberration of the return light from the fundus of the second measuring light.

3. The imaging apparatus according to claim 1, further comprising:
a detection unit configured to detect movement of the fundus; and
a third scanning unit, which is provided to the common optical system, and is arranged to change irradiation positions of the first measuring light and the second measuring light in order to correct the movement.

4. The imaging apparatus according to claim 1,
wherein the optical system includes:
a separation unit, which is arranged on the optical path extending to the second scanning unit, and is arranged to separate the first measuring light;
a reflection unit arranged to reflect the first measuring light separated by the separation unit; and
a joining unit arranged to join the first measuring light reflected by the reflection unit to the optical path extending from the second scanning unit, and
wherein the first measuring light is illuminated to the fundus by the separation unit and the joining unit via the first scanning unit without intermediation of the second scanning unit.

5. The imaging apparatus according to claim 4,
wherein the separation unit includes a beam splitter, and
wherein the joining unit includes a mirror.

6. The imaging apparatus according to claim 1, wherein the second scanning unit is driven at a frequency higher than a frequency for driving the first scanning unit.

7. The imaging apparatus according to claim 1, further comprising:
a first focus unit provided to the common optical system; and
a second focus unit provided on at least one of an optical path for the first measuring light or an optical path for the second measuring light, which is branched off from the common optical system.

8. An imaging apparatus configured to acquire an image of an imaging range of a fundus, the imaging apparatus comprising:
a first generating unit configured to generate a tomographic image of the fundus based on interference light obtained by causing return light from the fundus of first measuring light obtained by branching light emitted from a first light source, the return light being obtained when the first measuring light is scanned at a predetermined position in the imaging range, and reference light obtained by branching the light emitted from the first light source, to interfere with each other;
a second generating unit configured to generate a fundus image of the fundus based on return light from the fundus of second measuring light, the return light being obtained when the second measuring light is scanned over the imaging range; and
a correcting unit configured to correct irradiation positions of the first measuring light and the second measuring light on the fundus based on the fundus image generated by the second generating unit.

9. The imaging apparatus according to claim 8, further comprising:
a first scanning unit arranged to scan the first measuring light and the second measuring light over the imaging range in a first direction;
a second scanning unit arranged to scan the second measuring light over the imaging range in a second direction being a direction different from the first direction; and
a common optical system arranged to:
illuminate the fundus via the first scanning unit without intermediation of the second scanning unit with the first measuring light; and
illuminate the fundus via the first scanning unit and the second scanning unit with the second measuring light.

10. The imaging apparatus according to claim 9,
wherein the common optical system includes:
a wavefront sensor arranged to measure wavefront aberration; and
a wavefront correction apparatus configured to correct the wavefront aberration,
wherein the wavefront sensor is arranged to measure one of the wavefront aberration of the return light from the fundus of the first measuring light and the wavefront aberration of the return light from the fundus of the second measuring light, and
wherein the wavefront correction apparatus is configured to correct the wavefront aberration of the return light from the fundus of the first measuring light and the wavefront aberration of the return light from the fundus of the second measuring light.

11. The imaging apparatus according to claim 9,
wherein the common optical system includes:
a separation unit, which is arranged on the optical path extending to the second scanning unit, and is arranged to separate the first measuring light;
a reflection unit arranged to reflect the first measuring light separated by the separation unit; and
a joining unit arranged to join the first measuring light reflected by the reflection unit to the optical path extending from the second scanning unit, and
wherein the first measuring light is illuminated to the fundus by the separation unit and the joining unit via the first scanning unit without intermediation of the second scanning unit.

12. A control method for an imaging apparatus,
the imaging apparatus including:
a first scanning unit arranged to scan light on a fundus in a first direction;
a second scanning unit arranged to scan the light on the fundus in a second direction being a direction different from the first direction;
an optical system arranged to join an optical path extending to the second scanning unit to an optical path extending from the second scanning unit without intermediation of the second scanning unit;
a common optical system arranged to:
illuminate the fundus via the first scanning unit and the optical system with first measuring light obtained by branching light emitted from a first light source; and
illuminate the fundus via the first scanning unit and the second scanning unit with second measuring light emitted from a second light source;
a first generating unit configured to generate a tomographic image of the fundus based on interference light obtained by causing return light from the fundus of the first measuring light illuminated by the common optical system via the first scanning unit and the optical system, and reference light obtained by branching the light emitted from the first light source, to interfere with each other; and
a second generating unit configured to generate a fundus image of the fundus based on return light from the fundus of the second measuring light illuminated by the common optical system via the first scanning unit and the second scanning unit,
the control method comprising generating, by the first generating unit, the tomographic image at a predetermined position in the fundus image generated by the second generating unit.

13. A control method for an imaging apparatus configured to acquire an image of an imaging range of a fundus, the control method comprising:
a first generating step of generating a tomographic image of the fundus based on interference light obtained by causing return light from the fundus of first measuring light obtained by branching light emitted from a first light source, the return light being obtained when the first measuring light is scanned at a predetermined position in the imaging range, and reference light obtained by branching the light emitted from the first light source, to interfere with each other;
a second generating step of generating a fundus image of the fundus based on return light from the fundus of second measuring light, the return light being obtained when the second measuring light is scanned over the imaging range; and
a correcting step of correcting irradiation positions of the first measuring light and the second measuring light on the fundus based on the fundus image generated in the second generating step.

14. An imaging apparatus comprising:
an OCT optical system arranged to acquire tomographic information on an eye to be inspected using OCT measuring light;
an SLO optical system arranged to acquire fundus information on the eye to be inspected using SLO measuring light;
a common optical path to be used for the OCT optical system and the SLO optical system to share at least a part of an optical path for the OCT measuring light and an optical path for the SLO measuring light;
a first focus unit provided on the common optical path; and
a second focus unit provided on at least one of the optical path for the SLO measuring light or the optical path for the OCT measuring light, which is branched off from the common optical path.

15. The imaging apparatus according to claim 14, wherein the second focus unit has a focus adjustment range narrower than a focus adjustment range of the first focus unit.

16. The imaging apparatus according to claim 14, wherein the second focus unit is provided on the optical path for the SLO measuring light branched off from the common optical path.

17. The imaging apparatus according to claim 14, further comprising a control unit configured to control the first focus unit and the second focus unit.

18. The imaging apparatus according to claim 17, further comprising:
an aberration measuring unit arranged to measure aberration of the OCT measuring light; and
an aberration correcting unit, which is provided on the common optical path, and is configured to correct the aberration,
wherein the control unit is configured to control the aberration correcting unit based on the aberration measured by the aberration measuring unit.

19. The imaging apparatus according to claim 18, wherein the first focus unit comprises a Badal optical system formed of a reflective optical system provided on an optical path between the eye to be inspected and each of the aberration measuring unit and the aberration correcting unit.

20. The imaging apparatus according to claim 17, wherein the control unit is configured to control the second focus unit in linkage with the first focus unit.

21. The imaging apparatus according to claim 17, further comprising an optical path length adjusting unit provided on one of the optical path for the OCT measuring light and an optical path for reference light corresponding to the OCT measuring light in the OCT optical system,
wherein the control unit is configured to control the optical path length adjusting unit in linkage with the first focus unit.

22. The imaging apparatus according to claim 17, further comprising a scanning unit arranged to scan the OCT measuring light on the fundus of the eye to be inspected in a two-dimensional direction,
wherein the control unit is configured to:
detect movement of the fundus based on the fundus information on the eye to be inspected; and
control the scanning unit based on the detected movement of the fundus.

23. The imaging apparatus according to claim 22,
wherein the control unit is configured to detect the movement of the fundus by detecting a positional deviation between a reference image, which is a partial image of a planar image based on first fundus information acquired by the SLO optical system, and a target image, which is a partial image of a planar image based on second fundus information acquired by the SLO optical system after the first fundus information, and
wherein one of the reference image and the target image has an image size larger than an image size of another one of the reference image and the target image.

24. The imaging apparatus according to claim 17, further comprising:
a first scanning unit arranged to scan the OCT measuring light and the SLO measuring light in a first scanning direction;
a second scanning unit arranged to scan the OCT measuring light in a second scanning direction perpendicular to the first scanning direction; and
a third scanning unit arranged to scan the SLO measuring light in the second scanning direction,
wherein the control unit is configured to:
cause the first scanning unit to repeatedly scan the OCT measuring light and the SLO measuring light during a period in which the second scanning unit performs scanning one time; and
cause the third scanning unit to repeatedly scan the SLO measuring light during a period in which the first scanning unit performs scanning one time.

25. The imaging apparatus according to claim 24,
wherein the common optical path includes:
a first dichroic mirror arranged to separate the OCT measuring light and the SLO measuring light; and
a second dichroic mirror arranged to join the OCT measuring light and the SLO measuring light, which have been separated by the first dichroic mirror,
wherein the second scanning unit is arranged on the separated optical path for the OCT measuring light, and
wherein the third scanning unit is arranged on the separated optical path for the SLO measuring light.

26. The imaging apparatus according to claim 24, wherein at least one of the first scanning unit, the second scanning unit, or the third scanning unit is arranged between the eye to be inspected and the first focus unit.

27. The imaging apparatus according to claim 17, wherein the control unit is further configured to cause the first focus unit to adjust a focus state of the OCT measuring light and a focus state of the SLO measuring light, and then cause the second focus unit to adjust at least one of the focus state of the OCT measuring light or the focus state of the SLO measuring light.

28. The imaging apparatus according to claim 14, further comprising a linkage mechanism arranged to establish linkage between the second focus unit and the first focus unit to each other,
wherein the linkage between the second focus unit and the first focus unit is removable by the linkage mechanism.

29. The imaging apparatus according to claim 14, further comprising:
an optical path length adjusting unit provided on one of the optical path for the OCT measuring light and an optical path for reference light corresponding to the OCT measuring light in the OCT optical system; and
a linkage mechanism arranged to establish linkage between the optical path length adjusting unit and the first focus unit,
wherein the linkage between the optical path length adjusting unit and the first focus unit is removable by the linkage mechanism.
